SEQUENCE LISTING (2 sequences)

SEQ ID NO: 1 - protein, 112 aa, Homo sapiens [sequence omitted]

SEQ ID NO: 2 - DNA, 336 bp, Homo sapiens
  DNA encoding wild-type human TGF-Beta 3
gctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagc

(19) United States
(12) Patent Application Publication
(10) Pub. No.: US 2011/0166067 A1
(43) Pub. Date: Jul. 7, 2011

(54) METHODS FOR INHIBITION OF SCARRING

(75) Inventors: Mark William James Ferguson, Manchester (GB); Sharon O'Kane, Manchester (GB); Nicholas Occleston, Manchester (GB); Nicholas Goldspink, Manchester (GB); Kerry Nield, Manchester (GB); Abdul Sattar, Manchester (GB); James Bush, Manchester (GB)

(73) Assignee: RENOVO LIMITED, Manchester (GB)

(21) Appl. No.: 13/003,760

(22) PCT Filed: Dec. 11, 2008
(86) PCT No.: PCT/GB08/04078

§ 371 Date: Jan. 11, 2011

(30) Foreign Application Priority Data
Dec. 12, 2007 (GB) 0724204.3

(51) Int. Cl. A61K 38/18; C07K 14/495; A61P 17/02; A61P 9/00

(52) U.S. Cl. 514/8.9; 530/399

(57) ABSTRACT

The invention provides new methods of treatment using TGF-β3 to inhibit scarring in humans, and TGF-β3 for new uses in the inhibition of scarring in humans. In a first incidence of treatment TGF-β3 is provided to each centimetre of a wound margin or each centimetre of a site at which a wound is to be formed in a first therapeutically effective amount; and in a subsequent incidence of treatment TGF-β3 is provided to each centimetre of wound margin in a larger therapeutically effective amount of TGF-β3. The incidences of treatment occur between 8 hours and 48 hours apart from one another. The TGF-β3 may be provided by intradermal injection. Also provided are kits and methods of selecting an appropriate treatment regime for inhibiting scarring associated with the healing of a human wound.

The present invention relates to the provision of new methods for inhibiting scarring formed on healing of wounds. The invention also provides new uses of TGF-β3; new methods of selecting an appropriate treatment regime for inhibiting scarring associated with the healing of a wound; and kits for use in the inhibition of scarring associated with healing of a wound.

The Transforming Growth Factor-Betas (TGF-βs) are a family of cytokines having diverse biological activities. The TGF-β family comprises five isoforms, TGF-β1, TGF-β2, TGF-β3, TGF-β4, and TGF-β5. The members of the TGF-β family naturally exist in the form of dimers comprising two peptide chains. Active TGF-β dimers have a molecular weight of approximately 25.4 kDa.

TGF-β3 has been shown to be useful in the prevention, reduction or inhibition of scarring at sites throughout the body. This effect is particularly advantageous in relation to TGF-β3's ability to inhibit scarring associated with the healing of wounds. The amino acid sequence of human TGF-β3 is shown in Sequence ID No. 1, and the sequence of a nucleic acid encoding TGF-β3 is shown in Sequence ID No. 2.

The scarring response to healing of a wound is common throughout all adult mammals. The scarring response is conserved between the majority of tissue types and in each case leads to the same result, formation of fibrotic tissue termed a “scar”. A scar may be defined as “fibrous connective tissue that forms at the site of injury or disease in any tissue of the body”.

In the case of a scar that results from healing of a wound, the scar constitutes the structure produced as a result of the reparative response. This reparative process has arisen as the evolutionary solution to the biological imperative to prevent the death of a wounded animal. In order to overcome the risk of mortality due to infection or blood loss, the body reacts rapidly to repair the damaged area, rather than attempt to regenerate the damaged tissue. Since the damaged tissue is not regenerated to attain the same tissue architecture present before wounding, a scar may be identified by virtue of its abnormal morphology as compared to unwounded tissue.

Viewed macroscopically, scars may be depressed below the surface of the surrounding tissue, or elevated above the surface of their undamaged surroundings. Scars may be relatively darker coloured than normal tissue (hyperpigmentation) or may have a paler colour (hypopigmentation) compared to their surroundings. In the case of scars of the skin, either hyperpigmented or hypopigmented scars constitute a readily apparent cosmetic defect. It is also known that scars of the skin may be redder than unwounded skin, causing them to be noticeable and cosmetically unacceptable. It has been shown that the cosmetic appearance of a scar is one of the major factors contributing to the psychological impact of scars upon the sufferer, and that these effects can remain long after the wound that caused the scar has healed.

In addition to their psychological effects, scars may also have deleterious physical effects upon the sufferer. These effects typically arise as a result of the mechanical differences between scars and normal tissue. The abnormal structure and composition of scars mean that they are typically less flexible than their normal tissue counterpart. As a result scars may be responsible for impairment of normal function (such as in the case of scars covering joints which may restrict the possible range of movement) and may retard normal growth if present from an early age.

In the light of the above it will be recognised that TGF-β3 is of great utility in the clinical management of scarring that may occur on healing of wounds, but that there also remains a requirement for new and improved methods of treatment that may be used to inhibit scarring associated with the healing of wounds.

It is an object of some aspects of the present invention to provide improved methods of inhibiting scarring formed on healing of wounds. It is an object of other aspects of the invention to provide new uses of TGF-β3. These new uses of TGF-β3 may constitute alternative uses to those known from the prior art, but it may be preferred that they constitute improved uses compared to those already known. It is an object of certain aspects of the invention to provide; new methods of selecting an appropriate treatment regime for inhibiting scarring associated with the healing of a wound. It is an object of other aspects of the invention to provide kits for use in the inhibition of scarring associated with healing of a wound. These kits may be used in methods of treatment that provide increased inhibition of scarring compared to those known from the prior art.

In a first aspect of the invention there is provided a method of inhibiting scarring formed on healing of a wound, the method comprising treating a body site in which scarring is to be inhibited:

in a first incidence of treatment providing to each centimetre of wound margin, or each centimetre of a site at which a wound is to be formed a first therapeutically effective amount of TGF-β3; and
in a second incidence of treatment, occurring after a wound is formed and between 8 and 48 hours after the first incidence of treatment, providing to said wound a therapeutically effective amount of TGF-β3 that is larger than the therapeutically effective amount of TGF-β3 provided in the first incidence of treatment.

In a second aspect, the invention provides a method of inhibiting scarring formed on healing of a wound, the method comprising treating a body site in which scarring is to be inhibited:

in a first incidence of treatment providing to each centimetre of a site where a wound is to be formed a first therapeutically effective amount of TGF-β3; and
in a second incidence of treatment, occurring after a wound is formed and between 8 and 48 hours after the first incidence of treatment, providing to said wound a therapeutically effective amount of TGF-β3 that is larger than the therapeutically effective amount of TGF-β3 provided in the first incidence of treatment.

In a third aspect, the invention provides a method of inhibiting scarring formed on healing of a wound, the method comprising treating a body site in which scarring is to be inhibited:

in a first incidence of treatment providing to each centimetre of wound margin, or each centimetre of future wound margin, a first therapeutically effective amount of TGF-β3; and
in a second incidence of treatment, occurring after a wound is formed and between 8 and 48 hours after the first incidence of treatment, providing to said wound a therapeutically effective amount of TGF-β3 that is larger than the therapeutically effective amount of TGF-β3 provided in the first incidence of treatment.

The present invention is based upon the inventors' finding that scarring that would otherwise be expected on healing of a wound can be surprisingly effectively inhibited by use of a treatment regime, comprising at least two incidences of treatment, in which the site where scarring is to be reduced is treated with larger therapeutically effective amounts of TGF-β3 in the second (and any subsequent) incidence of treatment than in the first. The first incidence of treatment may occur at a time around wounding or wound closure, and then each further incidence of treatment may occur between 8 and 48 hours after the preceding incidence. These treatment regimes, described for the first time in the present disclosure, give rise to scars that are much reduced compared to those obtainable using known methods of treatment.

Without wishing to be bound by any hypothesis, the inventors believe that exposure of the cells at a wound or a site where a wound will be formed to the therapeutically effective amount of TGF-β3 provided in the first incidence of treatment is able to reduce the scarring response during the relatively early stages of wound healing. The TGF-β3 provided in the second (and any further) incidence of treatment may serve to counteract the pro-scarring “cascade” of biological processes that otherwise arises at the wound site. Such cascades are typically self-amplifying, with various pro-fibrotic factors capable of bringing about their own induction or the induction of further factors that induce scarring. The use of a larger dose of TGF-β3 in the second incidence of treatment appears to counteract this amplification, and thus inhibit scarring more effectively than can be achieved using the methods of the prior art.

It is important to note that this mode of treatment has not been suggested before, possibly as result of teachings of the prior art that are discussed below. However, the inventors have found that this new approach has a surprisingly beneficial effect in inhibiting scarring, which is noticeably greater than the effects that may be achieved using other TGF-β3 treatment regimes known to date.

The finding underlying the invention is highly surprising since not only are the anti-scarring results achieved particularly effective, but the prior art would have lead the skilled person to believe that this treatment regime using increasing doses of TGF-β3 would not be of as much benefit as known regimes using smaller doses.

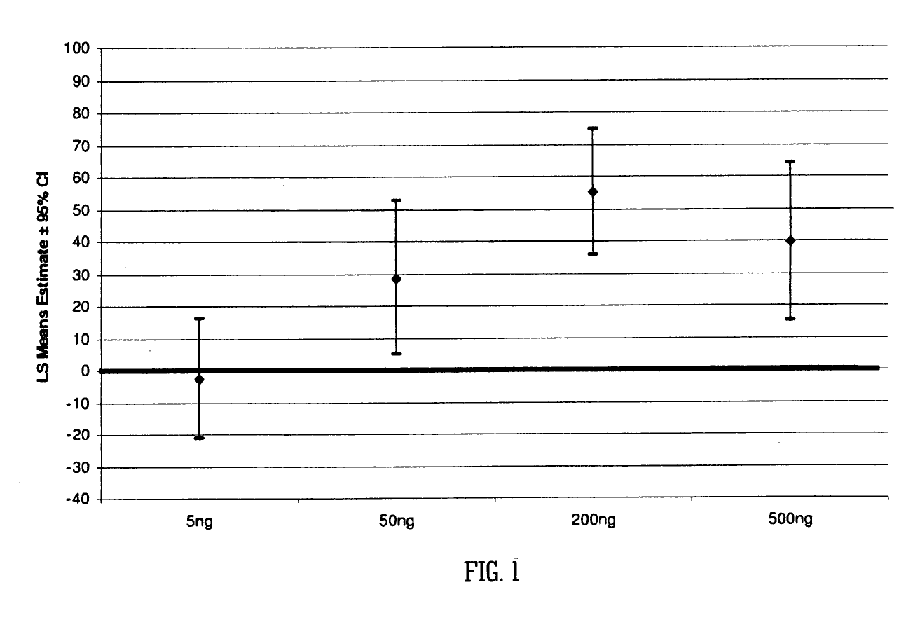

Previously it had been understood by those skilled in the art that the anti-scarring response to TGF-β3 took the form of a “bell shaped” dose response curve, of the sort shown in FIGS. 1, 2 and 10. Doses at the upper or lower ends of this curve were not as effective as those positioned in the middle of the dose response. Based on these findings a preferred therapeutically effective amount of TGF-β3 to be provided to a human patient per centimetre of a site in which scarring was to be inhibited had been identified as approximately 200 ng. Lower doses (of around 100 ng) or higher doses (such as 500 ng) did not give rise to such an effective reduction in scarring as did 200 ng. Furthermore studies conducted in animal models of scarring had suggested that elevated levels of TGF-β3 increase collagen in a way that may be expected to increase scarring. Investigations by the inventors, and by others working in this field, had determined that the 200 ng dose of TGF-β3 was effective in humans when administered prior to wounding, or to the wound margins after a wound is formed.

Once studies into the anti-scarring effectiveness of TGF-β3 had identified an optimal dose to be used for inhibition of human scarring as being 200 ng, further investigations considered whether any advantage was conferred by repeated administration of this dose to a site where scarring was to be reduced. These results showed that repeated administration of 200 ng TGF-β3 to wounds did not provide any benefits in terms of the anti-scarring effect observed.

Given that the dose response curve had identified that increasing the dose of TGF-β3 administered to a wound (in a single dose treatment regime) would reduce the effectiveness of the treatment, any suggestion to use escalating doses of TGF-β3 as part of a treatment regime would have been viewed as counterproductive. Based on the experiments that had been conducted (by the inventors and by other groups) it would have been anticipated that the use of multiple incidences of treatment would be no more effective than a single treatments regimes, but only more complex and expensive. Furthermore, it would have been expected that a regime in which the amount of TGF-β administered to a wound was increased over time would actually render treatment less effective than the favoured prior art regime (consisting of a single administration of a 200 ng dose) since it would cause the amount administered to rise into the upper portions of the bell-curve, where increasing dosage actively decreased anti-scarring effectiveness.

In the light of the above, it can be seen that the skilled person had no motivation to consider treatments of the sort described herein, in which repeated incidences of treatment are utilised, and the amount of TGF-β3 provided increases between the first and second treatments. Thus it will be appreciated that the findings set out in the present disclosure provide a surprising, but valuable, addition to the range of treatments that may be used to clinically inhibit the scarring of wounds.

Since the methods of treatment disclosed herein require at least two incidences of treatment, which take place between at least 8 to 48 hours apart from one another, they are not suitable for use in patients that would not be able to complete a second, or further incidence of treatment. This observation gives rise to a further aspect of the invention, in which there is provided a method of selecting an appropriate treatment regime for inhibiting scarring associated with the healing of a wound, the method comprising:
determining whether an individual in need of such inhibition of scarring will be able to complete a second incidence of treatment occurring between 8 and 48 hours after a first incidence of treatment; and
if the individual will be able to complete a second incidence of treatment occurring between 8 and 48 hours after a first incidence of treatment, selecting a treatment regime comprising a method of treatment in accordance with any of the first three aspects of the invention, or
if the individual will not be able to complete a second incidence of treatment occurring between 8 and 48 hours after a first incidence of treatment, selecting a treatment regime comprising:
in a single incidence of treatment providing to each centimetre of wound margin, or each centimetre of a site at which a wound is to be formed, in which scarring is to be inhibited an amount of between approximately 150 ng and 349 ng TGF-β3. Since the use in human patients is a preferred embodiment of this aspect of the invention, the amount of TGF-β3 provided per centimetre in this single incidence of treatment may preferably be approximately 200 ng.

In various aspects and embodiments of the invention, the present disclosure defines the amount of TGF-β3 to be provided to a body site with reference to the amount to be provided per centimetre of such a site (for example, per centimetre of a site to be wounded, or per centimetre of wound margin or of future wound margin). It will be appreciated that, while these passages define the amount of TGF-β3 to be provided to such sites, they do not limit the manner in which this amount is to be provided. In particular, these passages should not taken as requiring the administration of TGF-β3 to each centimetre of a site to be treated (though this may be a preferred embodiment). The requisite TGF-β3 may be provided by any number of administrations occurring at any site that allows the specified amount of TGF-β3 to be provided to the site at which scarring is to be inhibited.

In a further aspect of the invention there is provided TGF-β3 for use as a medicament in treating a wound or site where a wound is to be formed to inhibit scarring, wherein in a first incidence of treatment the medicament is provided such that a first therapeutically effective amount of TGF-β3 is provided to each centimetre of a wound margin or each centimetre of a site at which a wound is to be formed; and wherein in a subsequent incidence of treatment the medicament is provided such that a larger therapeutically effective amount of TGF-β3 is provided to each centimetre of a wound margin between 8 hours and 48 hours after the previous incidence of treatment.

A medicament in accordance with this aspect of the invention may be a re-constitutable medicament, such as a lyophilised injectable composition.

In another aspect the invention provides TGF-β3 for use as a medicament in treating a wound or site where a wound is to be formed to inhibit scarring, wherein in a first incidence of treatment the medicament is for provision such that a first therapeutically effective amount of TGF-β3 is provided to each centimetre of a wound margin or each centimetre of a site at which a wound is to be formed; and wherein in a subsequent incidence of treatment the medicament is for provision such that a larger therapeutically effective amount of TGF-β3 is provided to each centimetre of a wound margin between 8 hours and 48 hours after the previous incidence of treatment.

In a still further aspect of the invention there is provided TGF-β3 for inhibiting scarring formed on healing of a wound, wherein the TGF-β3 is prepared for administration in a first instance of treatment comprising providing to each centimetre of wound margin, or each centimetre of a site at which a wound is to be formed, a first therapeutically effective amount of TGF-β3 and in a second incidence of treatment, occurring after a wound is formed and between 8 and 48 hours after the first incidence of treatment, comprising providing to said wound a second therapeutically effective amount of TGF-β3 that is larger than the first therapeutically effective amount.

Furthermore, the inventors have found that the means for effecting the methods of the invention, including medicaments manufactured in accordance with the invention, may usefully be provided in the form of a kit for use in the inhibition of scarring associated with healing of a wound, the kit comprising at least first and second vials comprising TGF-β3 for administration to a wound, or a site where a wound is to be formed, at times between 8 and 48 hours apart from one another.

In a further aspect of the invention there is provided a kit for use in the inhibition of scarring associated with healing of a wound, the kit comprising:

a first amount of a composition containing TGF-β3, this first amount being for administration to a wound, or a site where a wound is to be formed, in a first incidence of treatment;
a second amount of a composition containing TGF-β3, this second amount being for administration to a wound in a second incidence of treatment;
instructions regarding administration of the first and second amounts of the composition at times between 8 and 48 hours apart from one another, and in a manner such that a larger therapeutically effective dose of TGF-β3 is administered to the wound in the second incidence of treatment than was administered in the first incidence of treatment.

A composition provided in such a kit may be provided in a form suitable for reconstitution prior to use (such as a lyophilised injectable composition).

It may be preferred that the first and second amounts of a composition respectively comprise different first and second compositions, wherein the second composition contains TGF-β3 at a greater concentration than does the first composition. In this case the instructions may indicate that a substantially similar volume of the first and second compositions should be administered to the site in the first and second incidences of treatment. Merely by way of example, the second composition may comprise TGF-β3 at a concentration that is approximately 100 ng/100 μl greater than the concentration in the first composition; or even 200 ng/100 μl, 500 ng/100 μl or 1000 ng/100 μl greater than the concentration in the first composition.

Alternatively, the first and second compositions may contain TGF-β3 at substantially equal concentrations, and the instructions may indicate that the volume of the second composition administered in the second incidence of treatment should be larger than the volume of the first composition administered in the first incidence of treatment.

In a further embodiment of the invention TGF-β3 is provided by a subcutaneous implant or depot medicament system for the pulsatile delivery of TGF-β3 to a wound or site where a wound is to be formed to inhibit scarring, wherein in a first incidence of treatment the medicament is provided such that a first therapeutically effective amount of TGF-β3 is provided to each centimetre of a wound margin or each centimetre of a site at which a wound is to be formed; and wherein in a subsequent incidence of treatment the medicament is provided such that a larger therapeutically effective amount of TGF-β3 is provided to each centimetre of a wound margin between 8 hours and 48 hours after the previous incidence of treatment.

A medicament in accordance with this aspect of the invention may be formulated in for example a bulk-eroding system such as polylactic acid and glycolic acid (PLGA) copolymer based microspheres or microcapsules systems containing the TGF-β3 or blends of PLGA:ethylcellulose systems may also be used. A further medicament in accordance with this aspect of the invention may be formulated in a surface-eroding system wherein the TGF-β3 is embedded in an erodible matrix such as the poly(ortho) ester and polyanhydride matrices wherein the hydrolysis of the polymer is rapid. A medicament in accordance with this aspect of the invention may also be formulated by combining a pulsatile delivery system as described above and an immediate release system such as a lyophilised injectable composition described above. It will be appreciated that, while TGF-β3 may be administered by the same route and in the same form in each incidence of treatment, different incidences of treatment may provide TGF-β3 by different medicaments and/or different routes of administration. In preferred embodiments of the invention the initial incidence of treatment may provide TGF-β3 by means of an injection, such as an intradermal injection, while the second (and any subsequent) incidences of treatment may involve provision of TGF-β3 by alternative routes, such as topical formulations.

The inventors believe that the benefits that may be derived from the present invention may be applicable to wounds at sites throughout the body. However, it may be preferred that the wound, scarring associated with which is to be inhibited, is a skin wound. For illustrative purposes the embodiments of the invention will generally be described with reference to skin wounds, although they remain applicable to other tissues and organs. Merely by way of example, in another preferred embodiment the wound may be a wound of the circulatory system, particularly of a blood vessel (in which case the treatments may inhibit restenosis). Other wounds in which scarring may be inhibited in accordance with the present invention are considered elsewhere in the specification. The wound may be a result of surgery (such as elective surgery), and this constitutes a preferred embodiment of the invention.

The inventors believe that the methods, uses and kits disclosed in the present specification may be used in the inhibition of scarring in all animals, including human or non-human animals, such as domestic animals, sporting animals (such as horses) or agricultural animals. Wounds in which scarring is to be inhibited will preferably be those of a human subject.

The methods of the invention may optionally comprise a third or further incidence of treatment. Such further incidences of treatment may be continued as necessary until a clinician responsible for the care of the patient determines that a desired inhibition of scarring has been achieved. Each incidence of treatment should occur between 8 and 48 hours after the preceding incidence of treatment. Further guidance as to timing of third or further incidences of treatment may be taken from the disclosure herein relating to the relative timing of the first and second incidences.

The amount of TGF-β3 provided to the body site in a third incidence of treatment (and any further incidence of treatment) may be substantially the same as the amount provided in the second incidence of treatment (thus the dose provided effectively “plateaus” after the second incidence of treatment). Alternatively, the amount of TGF-β3 provided to the body site in the third (or subsequent) incidence of treatment may be larger than the amount of TGF-β3 provided in the preceding incidence of treatment (so that the amount of TGF-β3 provided escalates with each incidence of treatment).

There are a number of ways in which the methods of treatment of the invention may be put into practice, and these will be apparent to those of skill in the art. Certain preferred embodiments will now be described below by way of non-limiting examples. It will be appreciated that these examples are applicable to each of the first three aspects of the invention.

In one embodiment the first and second incidences of treatment (and other incidences as appropriate) may both make use of a composition comprising TGF-β3 at substantially the same concentration. In this embodiment, the amount of the composition that is administered to the body site in the second incidence of treatment will be larger than the amount that is administered in the first incidence of treatment, and this difference provides the increase in dose between the different incidences.

It may be preferred that the first and second incidences of treatment (and, if appropriate any further incidences of treatment) make use of different compositions, wherein the composition used in the second incidence of treatment contains TGF-β3 at a greater concentration than does the composition used in the first incidence of treatment. In this case a substantially similar volume of the TGF-β3-containing compositions may be administered to the site in the first and second incidences of treatment (or even a smaller volume in the second incidence) since the increase in dose between the incidences occurs as a result of the increasing concentration of TGF-β3 in the compositions. Merely by way of example, the second (and further) incidences of treatment may make use of composition comprising TGF-β3 at a concentration that is approximately 100 ng/100 μl greater than the concentration in the composition used in the preceding incidence of treatment. Alternatively the concentrations of the compositions may differ by 200 ng/100 μl, 500 ng/100 μl or 1000 ng/100 μl more.

The therapeutically effective dose provided per centimetre of a body site (be it a site where a wound is to be formed, a wound margin, or a future wound margin) may comprise up to about 100 ng TGF-β3, up to about 200 ng TGF-β3, up to about 300 ng TGF-β3, up to about 400 ng TGF-β3, up to about 500 ng TGF-β3, up to about 600 ng TGF-β3, up to about 700 ng TGF-β3, up to about 800 ng TGF-β3, up to about 900 ng TGF-03, up to about 1000 ng TGF-β3, or more. Merely by way of example the amount of TGF-β3 administered per centimetre in the first incidence of treatment may be approximately 100 ng TGF-β3, 200 ng TGF-β3, 300 ng TGF-β3, 400 ng TGF-β3, 500 ng TGF-β3, 600 ng TGF-β3, 700 ng TGF-03, 800 ng TGF-β3, 900 ng TGF-β3, 1000 ng TGF-β3, or more. These values may be particularly suitable for embodiments concerned with the inhibition of scarring in human patients.

The therapeutically effective dose provided per centimetre of body site in the second incidence of treatment may be approximately 100 ng TGF-β3, 200 ng TGF-β3, 300 ng TGF-β3, 400 ng TGF-β3, 500 ng TGF-β3, 600 ng TGF-β3, 700 ng TGF-β3, 800 ng TGF-β3, 900 ng TGF-β3, or even 1000 ng TGF-β3 greater than the dose in the first incidence. Subsequent incidences of treatment may use a dose of TGF-β3 that varies from that provided in the previous incidence of treatment by approximately 100 ng TGF-β3, 200 ng TGF-β3, 300 ng TGF-β3, 400 ng TGF-β3, 500 ng TGF-β3, 600 ng TGF-β3, 700 ng TGF-β3, 800 ng TGF-β3, 900 ng TGF-β3 or 1000 ng TGF-β3. These values may be particularly suitable for embodiments concerned with the inhibition of scarring in human patients.

It will be appreciated that although the amount of TGF-β3 to be provided in each incidence of treatment is referred to in the present disclosure on the basis of the amount to be provided per centimetre, the disclosure is not limited by this, and may be used to determine suitable doses that may be applied to a wound measured by any suitable unit

It may be preferred that the first incidence of treatment occurs prior to wounding, in which case TGF-β3 may be provided to a site where a wound is to be formed. In the case that the TGF-β3 is administered by local injection to the skin (such as intradermal injection) this may cause a bleb to be raised as a result of the introduction of a TGF-β3 containing solution into the skin. In one preferred embodiment the bleb may be raised in the site where the wound is to be formed, and indeed the wound may be formed by incising the bleb. In this case the amount of TGF-β3 to be provided in the first incidence of treatment may be determined with reference to the length of the site where the wound is to be formed.

Alternatively two blebs may be raised, on either side of the site where the wound is to be formed. These blebs may preferably be positioned within half a centimetre of where the margins of the wound will be formed. In this case the amount of TGF-β3 to be provided in the first incidence of treatment may be determined with reference to the length of the wound to be formed, measured in centimetres of future wound margin (defined below).

Preferably a bleb used to provide TGF-β3 to a site prior to wounding may cover substantially the full length of the site where the wound is to be formed. More preferably the bleb may extend beyond the length of the site where a wound is to be formed. Suitably such a bleb may extend around half a centimetre (or more) beyond each end of the wound to be formed.

Intradermal injections in accordance with these embodiments of the invention may be administered by means of a hypodermic needle inserted substantially parallel to the midline of the wound to be formed, or parallel to the margins of the wound to be formed. Injection sites may be spaced approximately one centimetre apart from one another along the length of the region to which TGF-β3 will be provided.

In the alternative, it may be preferred that the first incidence of treatment involves provision of TGF-β3 to an existing wound. The inventors believe that the biological mechanisms relevant to the anti-scarring activity are the same whether cells are exposed to TGF-β3 before or after wounding. In either case, the necessary biological activity may be achieved as long as the cells at the site where scarring is to be inhibited are exposed to a therapeutically effective amount of between approximately 100 and 1000 ng TGF-β3 either before or after wounding.

In embodiments of the invention in which TGF-β3 is to be provided to an existing wound, the requisite amount may be determined with reference to the length of the wound, measured in centimetres of wound margin (as discussed below). TGF-β3 should preferably be provided along the entire length of each wound margin, and may even be provided beyond the wounded area. In a preferred embodiment TGF-β3 may be provided along a length extending about half a centimetre (or more) beyond the ends of the margins of the wound.

Intradermal injection also represents a preferred route by which TGF-β3 may be administered to an existing wound. Intradermal injections administered in accordance with this embodiment should be administered to each margin of the wound. The site of injection may preferably be within half a centimetre of the edge of the wound. The injections may be administered by means of a hypodermic needle inserted substantially parallel to the edge of the wound. Injection sites may be spaced approximately one centimetre apart from one another along the length of the region to be treated.

The considerations set out in the preceding paragraphs in relation to provision of TGF-β3 to a wound in the first incident of treatment will also be applicable to its provision in second (or further) incidents. Since the second incidence of treatment takes place after wounding has occurred this will always involve provision of TGF-β3 to an existing wound. The wound may be open or closed, depending on the wound management strategy that is being applied.

When the first incidence of treatment involves provision of TGF-β3 to a site where a wound is to be formed it may be preferred that this provision occurs an hour or less before wounding is initiated, preferably half an hour or less before wounding is initiated, still more preferably a quarter of an hour or less before wounding is initiated, and most preferably ten minutes or less before wounding is initiated.

If the first incidence of treatment is to involve provision of TGF-β3 to an existing wound, the time at which this treatment is provided may be selected with reference to time elapsed after the wound has been formed. In this case, it may be preferred that a first incidence of treatment in accordance with the invention is initiated within two hours of wounding, preferably within one and a half hours of wounding, more preferably within an hour of wounding, still more preferably within half an hour of wounding, and most preferably within a quarter of an hour of wounding.

Alternatively or additionally, the timing of the first incidence of treatment may be selected with reference to the time elapsed after closure of the wound to be treated. In this case, it may be preferred that a first incidence of treatment in accordance with the invention is initiated within two hours of the closure of the wound being completed, preferably within one and a half hours of closure of the wound being completed, more preferably within an hour of closure of the wound being completed, still more preferably within half an hour of closure of the wound being completed, and most preferably within a quarter of an hour of closure of the wound being completed. In the case that a wound is not to be completely closed for clinical reasons (for example if it is necessary to maintain access to a site within the wound) closure of the wound may still be considered to have been completed once the wound is closed to the fullest extent that will be closed as part of the procedure undertaken.

It will be appreciated that selection of the timing of the first incidence of treatment with reference to the time elapsed after closure of the wound may be of particular relevance in the case of protracted surgical procedures, where a wound must be kept open for a prolonged time in order to allow access to a site where surgery is being performed.

The time elapsing between incidences of treatment will be between 8 and 48 hours. More preferably the time elapsing should be at least, more preferably at least 10 hours, even more preferably at least 12 hours, yet more preferably at least 14 hours, still more preferably at least 16 hours, yet more preferably still at least 18 hours, more preferably still 20 at least hours, ever more preferably at least 22 hours, and most preferably is approximately 24 hours.

The time elapsing between incidences of treatment may be up to 48 hours, but will preferably be up to approximately 44 hours, more preferably up to approximately 40 hours, even more preferably up to approximately 36 hours, yet more preferably up to approximately 32 hours, still more preferably up to approximately 28 hours, and most preferably is approximately 24 hours.

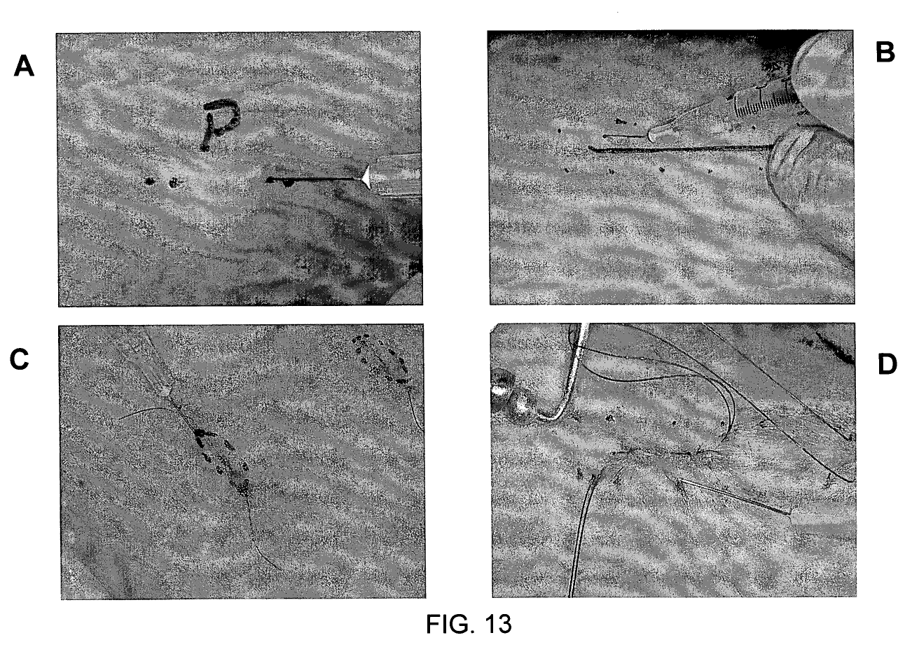

In practicing the methods of the invention, the cells of the area in which scarring is to be inhibited should be “bathed” in a pharmaceutically acceptable solution comprising a therapeutically effective amount of TGF-β3. This will create a local environment in which the cells are exposed to sufficient TGF-β3 to prevent scarring. Cells that would otherwise be involved in scar formation will receive the therapeutically effective amount of TGF-β3 whether the TGF-β3 is administered by injection at the margins of a wound (or along the margins of a future wound—technique shown in panel B of FIG. 13), or by injection directly into the site at which the wound is to be formed (for example, by raising a bleb covering the site to be wounded—technique shown in panel A of FIG. 13). Either of these routes of administration are able to establish an anti-scarring concentration of TGF-β3 in the area surrounding the cells.

When the first incidence of treatment utilises injection directly into the site to be wounded, the requisite amount of TGF-β3 may be established around the cells by administration of a single injection (or series of “single” injections) administered along the line of the future wound and which cover the area to be wounded (technique illustrated in panel A of FIG. 13). When the first incidence of treatment utilises “paired” injections to each margin of a wound (or “paired” injections down each future margin of a wound—technique illustrated in panel B of FIG. 13) it will be appreciate that the total amount of TGF-β3 to be administered will be larger than that provided via the single injection route (described above), since injections on each margin are required in order to treat the same area.

It is preferred that TGF-β3 be provided to the requisite body site in the methods of the invention by means of an administration of a suitable pharmaceutical composition. Generally, any pharmaceutically acceptable solution may be used, but the inventors have found that compositions for use in accordance with the invention may advantageously comprise a sugar such as maltose or trehalose. Such sugars may serve to stabilise the composition, and also increase the biological activity of TGF-β3 so compounded. Preferred compositions may be those suitable for injection, and in particular for intradermal injection. Many formulations of compositions that may be used for the administration of TGF-β3 by intradermal injection will be known to those skilled in the art. Examples of suitable formulations are described in the inventors' co-pending patent application, published as WO 2007/007095, and formulations of the sort described in this application were used in the studies reported in the Experimental Results section of the present specification.

Various terms used in the present disclosure will now be described further for the avoidance of doubt. It will be appreciated that, for the sake of brevity, some of these terms may be described with reference to only certain aspects of the invention. However, except for where the context requires otherwise, the following descriptions of these terms will be applicable to all aspects of the invention.

### Calculation of TGF-β3 Content, Potency and Amounts Administered

The protein content of solutions containing TGF-β3 (and in particular recombinant human TGF-β3, which is a preferred form of TGF-β3 to be used in accordance with the invention) may preferably be determined by quantitative Enzyme-linked Immunosorbent Assay (ELISA) calibrated with the United Kingdom National Institute for Biological Standards and Control (NIBSC) Transforming Growth Factor Beta-3 (Human rDNA derived) Reference Reagent code 98/608. Determination of protein content in this manner allows the concentration of solutions, and thus the amount of TGF-β3 that will be provided to a centimetre of a body site by a given volume of a solution, to be calculated by the skilled person. This protocol has been used in determining the protein content of solutions used in the Experimental Results section.

In the event that a skilled person is not able to obtain a reference sample of NIBSC Reference Reagent code 98/608, the inventors have found that ELISAs using their own TGF-β3 product (Lonza Bulk Drug Substance Lot 205-0505-005) as a standard give rise to values that are approximately 52% of those obtained with NIBSC Reference Reagent code 98/608. In the event that it is wished to use this alternative standard, instead of NIBSC Reference Reagent code 98/608, required amounts of TGF-β3 should be determined accordingly.

The biological activity (i.e. potency) of TGF-β3 to be used in accordance with the present invention may be determined by the inhibition of proliferation of Mink Lung Epithelial Cell line (MLEC); American Type Culture Collection (ATCC) Cat No. CCL-64. In a preferred embodiment, biological activity may be quantified by means of an assay calibrated using the United Kingdom National Institute for Biological Standards and Control Reference Reagent code 98/608, referred to above. Reference Reagent code 98/608 is considered to have a specific biological activity of 10 000 Arbitrary Units (AU) per microgram of TGF-β3 protein, and, by comparing the MLEC-inhibitory-activity of a sample of interest with the MLEC-inhibitory-activity of Reference Reagent code 98/608, the biological activity of the sample of interest in AU can be readily determined.

Thus a dose of 500 ng of TGF-β3 provides 5,000 AU of TGF-β3 activity, and a dose of 1000 ng of TGF-β3 provides 10,000 AU of TGF-β3 activity. The inventors believe that a similar therapeutic effect may be achieved by an amount of TGF-β3 activity between approximately 3,500 and 6,500 AU in the case of a 500 ng dose, and between 8,500 and 11,500 AU in the case of a 1000 ng dose. References to the use of doses of 500 ng, 1000 ng, or the like, of TGF-β3 in the present disclosure may be construed accordingly.

Centimetre of a Site where a Wound is to be Formed

For ease of reference, the length of a site where a wound is to be formed may be measured in centimetres in order to determine the amount of TGF-β3 that will need to be provided in order to reduce scarring in accordance with the invention. It may be preferred that the length to be treated be calculated to extend beyond the intended length of the wound to be formed, in order to ensure that a therapeutically effective amount of TGF-β3 is provided to the ends of the wound. Accordingly, it may be preferred that the calculated length of a site where a wound is to be formed (and hence the length of the site to be treated) extend by a distance of about half a centimetre (or more) beyond each end of the intended wound.

### Centimetre of Future Wound Margin

For the purposes of the present disclosure the length of a site where a wound is to be formed, as measured in number of centimetres of future wound margin, should be calculated as the sum of the lengths of each margin of the wound to be formed (in centimetres). It may be preferred that the length to be treated be calculated to extend beyond the ends of the margins of the wound to be formed, and this may help to ensure that a therapeutically effective amount of TGF-β3 is provided to the ends of the wound. Accordingly, it may be preferred that the calculated length of a future wound margin (and hence the length of the site to be treated) extend by a distance of about half a centimetre (or more) at each end of the wound to be formed.

### Centimetre of Wound Margin

For the purposes of the present disclosure, the length of a wound, as measured in number of centimetres of wound margin, should be calculated as the sum of the lengths of each margin of the wound (in centimetres). It may be preferred that the length of the site to be treated be calculated to extend beyond the ends of the margins of the wound. This may help to ensure that a therapeutically effective amount of TGF-β3 is provided to the ends of the wound. Accordingly, it may be preferred that the calculated length of a wound margin to be treated in accordance with the invention extend by a distance of about half a centimetre (or more) beyond each end of the wound.

### TGF-β3

For the purposes of the present disclosure, TGF-β3 may be taken to comprise a peptide comprising the amino acid sequence shown in Sequence ID No. 1. The TGF-β3 may preferably be dimeric TGF-β3, but the inventors believe that the inhibition of scarring described herein may also be achieved using monomeric forms of TGF-β3. It is the homodimeric active fragment of wild type human TGF-β3 (comprising two polypeptide chains each having the sequence of amino acid residues shown in Sequence ID No. 1) that was used in the studies described in the Experimental Results section.

The inventors believe that the inhibition of scarring described in the present disclosure may also be achieved using therapeutically effective fragments or derivatives of TGF-β3. Fragments of TGF-β3 may readily be determined with reference to the sequence information provided in Sequence ID No. 1, and derivatives may be prepared based on this sequence information using means well known to those skilled in the art. Examples of suitable derivatives are disclosed in the inventors' co-pending application published as WO2007/104845.

In the event that it is desired to use forms of TGF-β3 other than the wild type dimeric active fragment (comprising two peptide chains corresponding to Sequence ID No.1), it will be appreciated that such agents may have molecular weights that are not the same as that of the naturally occurring form. Accordingly, the therapeutically effective amounts of such agents to be used in the medicaments or methods of the invention may be varied, to reflect the differences in molecular weights. Thus, if a form of TGF-β3 having half the molecular weight of the wild type dimeric active fragment is to be used, then a suitable therapeutically effective amount will be half those set out elsewhere in the specification.

The therapeutic effectiveness of such fragments or derivatives of TGF-β3 may be readily assessed with reference to any one of a number of suitable experimental models. Such models may include in vitro models indicative of biological effectiveness (which may be expected to correlate with therapeutic effectiveness), or in vivo studies using human or non-human subjects. Merely by way of example, the techniques described in the Experimental Results section set out elsewhere in the specification may be used or adapted in order to investigate therapeutic effectiveness of fragments or derivatives of TGF-β3.

### “Therapeutically Effective Amounts”

A therapeutically effective amount of TGF-β3 for the purposes of the present disclosure is any amount of TGF-β3 that is able to prevent, reduce or inhibit scarring associated with healing of a wound when used in accordance with the present invention. It will be appreciated that amounts of TGF-β3 that are not therapeutically effective when considered in, for example, dose response experiments using single administrations of TGF-β3 may still be therapeutically effective in a model of scarring using two incidences of treatment, as described in the present specification.

### Prevention/Inhibition/Reduction/Minimisation of Scarring

The inhibition of scarring within the context of the present invention should be understood to encompass any degree of prevention, reduction, minimisation or inhibition in scarring achieved on healing of a wound treated in accordance with a method of the invention (or a kit or medicament of the invention) as compared to the level of scarring occurring on healing of a control-treated or untreated wound. For the sake of brevity, the present specification will primarily refer to “inhibition” of scarring utilising TGF-β3, however, such references should be taken, except where the context requires otherwise, to also encompass the prevention, reduction or minimisation of scarring using TGF-β3.

### Pharmaceutically Acceptable

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the US Federal or a state government or listed in the U.S. Pharmacopoeia or other generally recognized pharmacopeias for use in animals, and more particularly in humans.

### Pharmaceutical Compositions and Administration

While it is possible to use a composition provided by the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. Accordingly, in one aspect, the present invention provides a pharmaceutical composition or formulation comprising at least one active composition, or a pharmaceutically acceptable derivative thereof, in association with a pharmaceutically acceptable excipient, diluent and/or carrier. The excipient, diluent and/or carrier must be “acceptable” in the sense of being compatible with the other ingredients of the formulation and not deleterious to the recipient thereof.

The compositions of the invention can be formulated for administration in any convenient way for use in human or veterinary medicine. The invention therefore includes within its scope pharmaceutical compositions comprising a product of the present invention that is adapted for use in human or veterinary medicine.

Acceptable excipients, diluents, and carriers for therapeutic use are well known in the pharmaceutical art, and are described, for example, in Remington: The Science and Practice of Pharmacy. Lippincott Williams & Wilkins (A.R. Gennaro edit. 2005). The choice of pharmaceutical excipient, diluent, and carrier can be selected with regard to the intended route of administration and standard pharmaceutical practice.

### Wounds

The inventors believe that methods of treatment using TGF-β3 in accordance with the present invention may be used to beneficially inhibit scarring in all types of wounds.

Examples of specific wounds in which scarring may be inhibited using the medicaments and methods of the invention include, but are not limited to, those independently selected from the group consisting of: wounds of the skin; wounds of the eye (including the inhibition of scarring resulting from eye surgery such as LASIK surgery, LASEK surgery, PRK surgery, glaucoma filtration surgery, cataract surgery, or surgery in which the lens capsule may be subject to scarring) such as those giving rise to corneal cicatrisation; wounds subject to capsular contraction (which is common surrounding breast implants); wounds of blood vessels; wounds of the central and peripheral nervous system (where prevention, reduction or inhibition of scarring may enhance neuronal reconnection and/or neuronal function); wounds of tendons, ligaments or muscle; wounds of the oral cavity, including the lips and palate (for example, to inhibit scarring resulting from treatment of cleft lip or palate); wounds of the internal organs such as the liver, heart, brain, digestive tissues and reproductive tissues; wounds of body cavities such as the abdominal cavity, pelvic cavity and thoracic cavity (where inhibition of scarring may reduce the number of incidences of adhesion formation and/or the size of adhesions formed); and surgical wounds (in particular wounds associated with cosmetic procedures, such as scar revision). It is particularly preferred that the medicaments and methods of the invention be used to prevent, reduce or inhibit scarring associated with wounds of the skin.

### Assessment of Scarring

The extent of scarring, and so any inhibition of scarring achieved, may be assessed by macroscopic clinical assessment of scars. This may be achieved by the direct assessment of scars upon a subject; or by the assessment of photographic images of scars; or of silicone moulds taken from scars, or positive plaster casts made from such moulds. For the purposes of the present disclosure a “treated scar” should be taken to comprise a scar produced on healing of a wound treated in accordance with the present invention.

Macroscopic characteristics of a scar which may be considered when assessing scarring include:
i) Colour of the scar. Scars may typically be hypopigmented or hyperpigmented with regard to the surrounding skin. Inhibition of scarring may be demonstrated when the pigmentation of a treated scar more closely approximates that of unscarred skin than does the pigmentation of an untreated scar. Scars may often be redder than the surrounding skin. In this case inhibition of scarring may be demonstrated when the redness of a treated scar fades earlier, or more completely, or to resemble more closely the appearance of the surrounding skin, compared to an untreated scar. Colour can readily be measured, for example by use of a spectrophotometer.
ii) Height of the scar. Scars may typically be either raised or depressed as compared to the surrounding skin. Inhibition of scarring may be demonstrated when the height of a treated scar more closely approximates that of unscarred skin (i.e. is neither raised nor depressed) than does the height of an untreated scar. Height of the scar can be measured directly on the patient (e.g. by means of profilometry), or indirectly, (e.g. by profilometry of moulds taken from a scar).
iii) Surface texture of the scar. Scars may have surfaces that are relatively smoother than the surrounding skin (giving rise to a scar with a “shiny” appearance) or that are rougher than the surrounding skin. Inhibition of scarring may be demonstrated when the surface texture of a treated scar more closely approximates that of unscarred skin than does the surface texture of an untreated scar. Surface texture can also be measured either directly on the patient (e.g. by means of profilometry), or indirectly (e.g. by profilometry of moulds taken from a scar).
iv) Stiffness of the scar. The abnormal composition and structure of scars means that they are normally stiffer than the undamaged skin surrounding the scar. In this case, inhibition of scarring may be demonstrated when the stiffness of a treated scar more closely approximates that of unscarred skin than does the stiffness of an untreated scar.

A treated scar will preferably exhibit inhibition of scarring as assessed with reference to at least one of the parameters for macroscopic assessment set out in the present specification. More preferably a treated scar may demonstrate inhibited scarring with reference to at least two of the parameters, even more preferably at least three of the parameters, and most preferably at least four of these parameters (for example, all four of the parameters set out above).

The height, length, width, surface area, depressed and raised volume, roughness/smoothness of scars can be measured directly upon the subject, for example by using an optical 3D measurement device. Scar measurements can be made either directly on the subject, or on moulds or casts representative of the scar (which may be formed by making a silicone mould replica impression of the scar and subsequently creating a plaster cast from the silicone moulds). All of these methods can be analysed using an optical 3D measurement device, or by image analysis of photographs of the scar. 3D optical measurements have a resolution in the micrometer range along all axes which guarantees a precise determination of all skin and scar parameters. The skilled person will also be aware of further non-invasive methods and devices that can be used to investigate suitable parameters, including calipers for manual measurements, ultrasound, 3D photography (for example using hardware and/or software available from Canfield Scientific, Inc.) and high resolution Magnetic Resonance Imaging.

Inhibition of scarring may be demonstrated by a reduction in the height, length, width, surface area, depressed or raised volume, roughness or smoothness or any combination thereof, of a treated scar as compared to an untreated scar.

One preferred method for the macroscopic assessment of scars is holistic assessment. This may be accomplished by means of assessment of macroscopic photographs by an expert panel or a lay panel, or clinically by means of a macroscopic assessment by a clinician or by patients themselves. Assessments may be captured by means of a VAS (visual analogue scale) or a categorical scale. Examples of suitable parameters for the assessment of scarring (and thereby of any reduction of scarring attained) are described below. Further examples of suitable parameters, and means by which assessment of such parameters may be captured, are described by Duncan et al. (2006), Beausang et al. (1998) and van Zuijlen et al. (2002).

### Assessment Using Visual Analogue Scale (VAS) Scar Scores.

Assessments of scars may be captured using a scarring-based VAS. A suitable VAS for use in the assessment of scars may be based upon the method described by Duncan et al. (2006) or by Beausang et al. (1998). This is typically a 10 cm line in which 0 cm is considered an imperceptible scar and 10 cm a very poor hypertrophic scar. Use of a VAS in this manner allows for easy capture and quantification of assessment of scarring. VAS scoring may be used for the macroscopic and/or microscopic assessment of scarring.

Merely by way of example, a suitable macroscopic assessment of scarring may be carried out using a VAS consisting of a 0-10 cm line representing a scale, from left to right, of 0 (corresponding to normal skin) to 10 (indicative of a bad scar). A mark may be made by an assessor on the 10 cm line based on an overall assessment of the scar. This may take into account parameters such as the height, width, contour and colour of the scar. The best scars (typically of small width, and having colour, height and contour like normal skin) may be scored towards the “normal skin” end of the scale (the left hand side of the VAS line) and bad scars (typically large width, raised profile and with uneven contours and whiter colour) may be scored towards the “bad scar” end of the scale (the right hand side of the VAS line). The marks may then be measured from the left hand side to provide the final value for the scar assessment in centimetres (to 1 decimal place).

An alternative assessment of scarring (whether macroscopic assessment or microscopic assessment), involving the comparison of two scars or two scar segments (such as one treated segment and another segment untreated, or control treated) to determine which one has a preferred appearance, may be carried out using a VAS comprising two 100 mm VAS lines intersected by a vertical line. In a VAS of this sort, the two VAS lines correspond to the two scars being compared, while the vertical line represents zero (indicating that there is no perceptible difference between the scars compared). The extremes of 100% (100 mm at the end of either VAS line) indicate one of the scars has become imperceptible in comparison to the surrounding skin.

A particularly preferred method of assessing the macroscopic appearance of scars in this manner is referred to as The Global Scar Comparison Scale (GSCS). This scale has been positively received by the European Medicines Agency (EMEA) and accepted as a preferred scale by which scars may be assessed and clinically relevant endpoints associated with the inhibition of scarring determined. In particular, it may be preferred to use a version of the GSCS based on clinical panel assessment, this being viewed by the EMEA as particularly relevant.

When comparing a pair of scars using a VAS of this sort, such as the GSCS, an assessor first determines which of the scars has the preferred appearance, or if there is no perceptible difference between the two. If there is no perceptible difference this is recorded by placing a mark at the zero vertical line. If there is a perceptible difference, the assessor uses the worse of the two scars as an anchor to determine the level of improvement found in the preferred scar, and then marks the score on the relevant section of the scale. (i.e, setting a scale according to the comparison of scar appearance). The point marked represents the percentage improvement over the anchor scar.

The inventors have found that use of VAS measures of this sort in assessing the macroscopic or microscopic appearance of scars offers a number of advantages. Since these VAS are intuitive in nature they, 1) reduce the need for extensive training using reference images of different scar severities in different skin types, making this tool relatively simple to deploy in a large phase 3 trial; 2) reduce variability of the data: one assessment of each scar pair is performed as opposed to two independent assessments of drug and placebo scars; 3) incorporate the well-established principles of VAS (i.e., a continuous distribution of data) and the benefits of ranking in the same scale; and 4) allow easier communication of drug effect (percentage improvement) to clinicians and patients.

The present invention will now be further described with reference to the accompanying Experimental Results section and Figures, in which:

FIG. 1 compares the anti-scarring activity of different doses of TGF-β3 provided to human wounds in a single incidence of treatment.

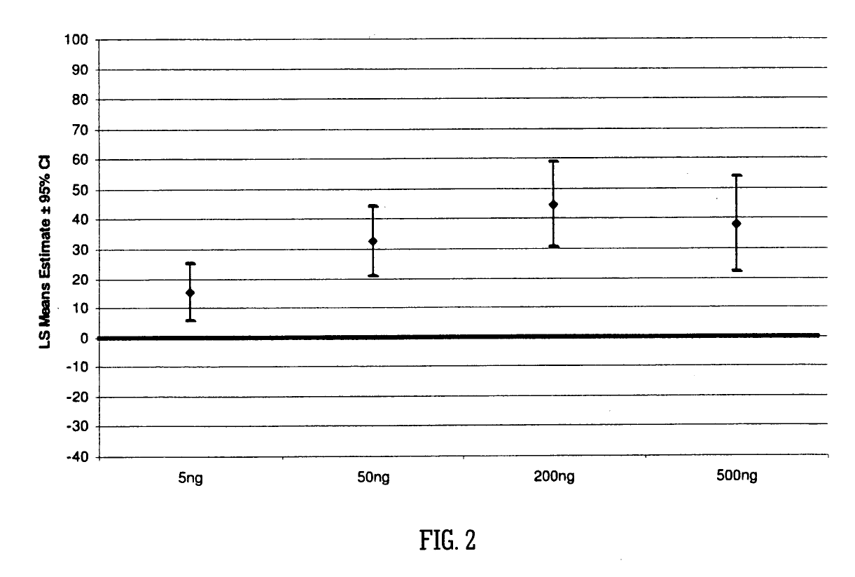

FIG. 2 compares the anti-scarring activity of different doses of TGF-β3 provided to human wounds in two incidences of treatment administered within approximately one hour of one another.

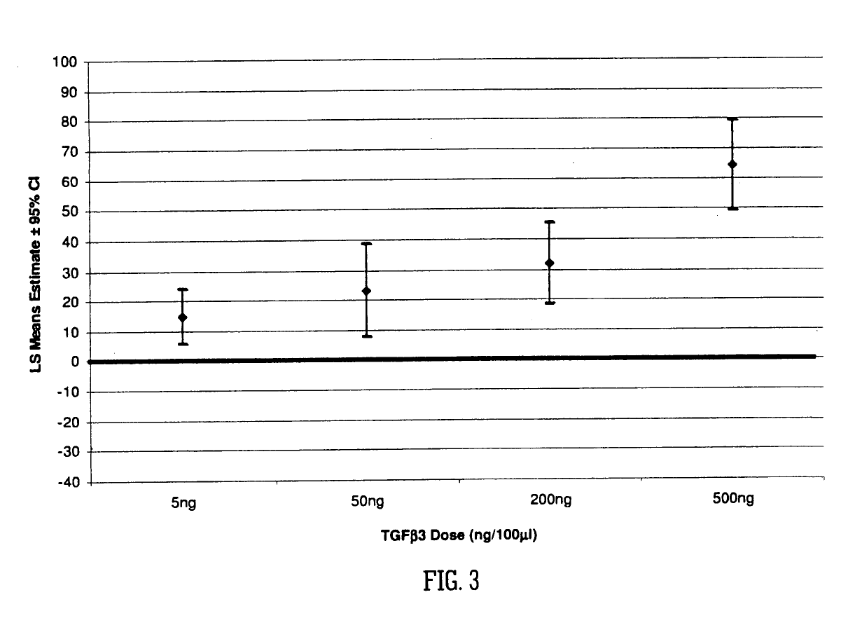

FIG. 3 compares the anti-scarring activity of different doses of TGF-β3 provided to human wounds in two incidences of treatment administered approximately 24 hours apart from one another.

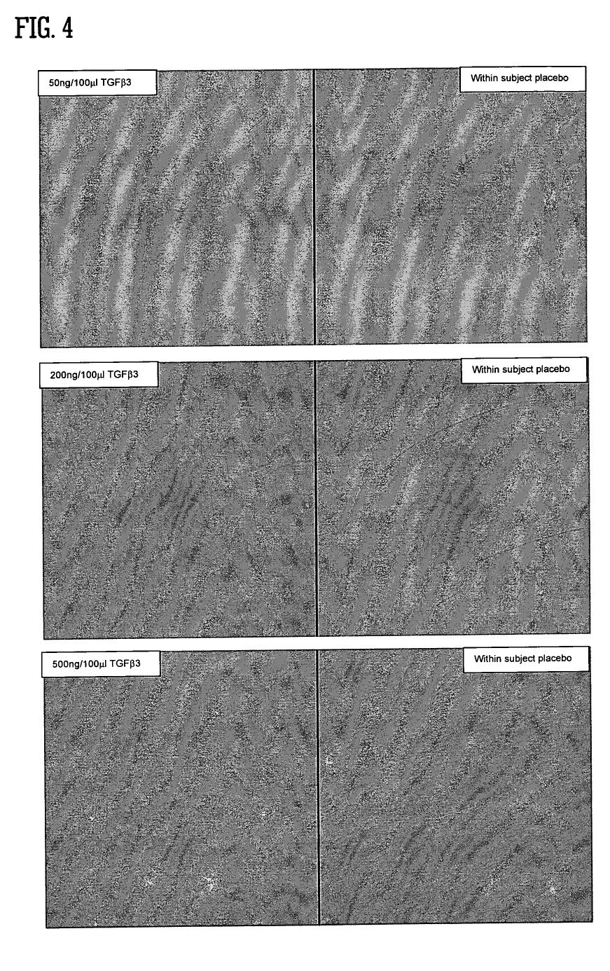

FIG. 4 compares macroscopic images of TGF-β3 control treated scars or placebo treated control scars. The three TGF-β3-treated scars were provided with different amounts of TGF-β3 in incidences of treatment separated by about 24 hours.

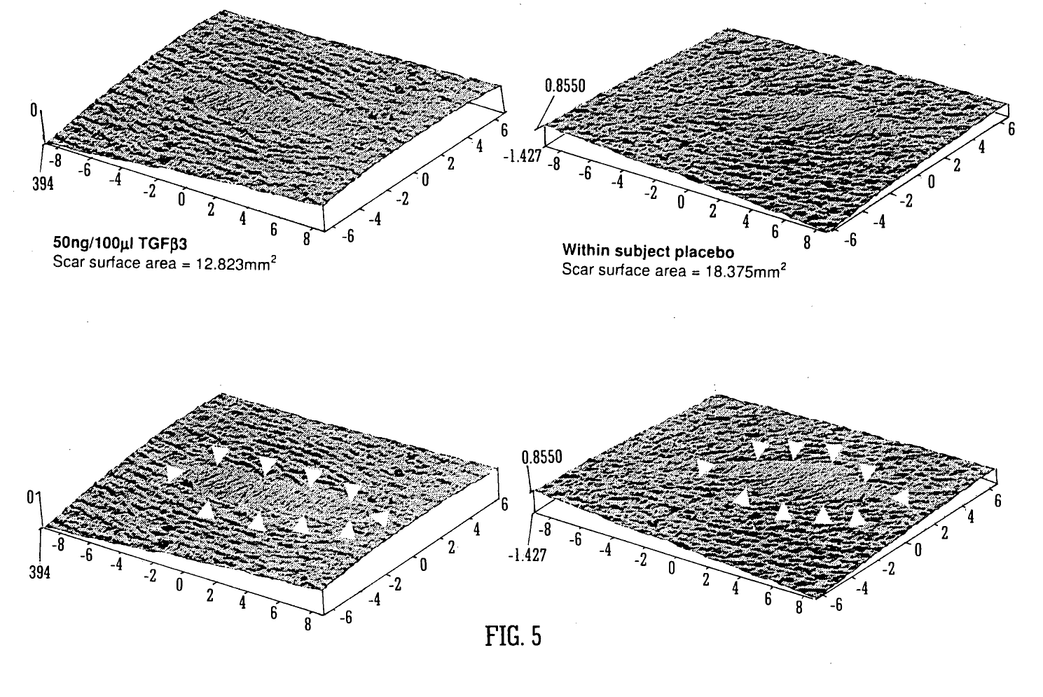

FIG. 5 illustrates 3-dimensional simulations and scar measurements taken from scars formed on healing of wounds treated with either TGF-β3 controls or placebo.

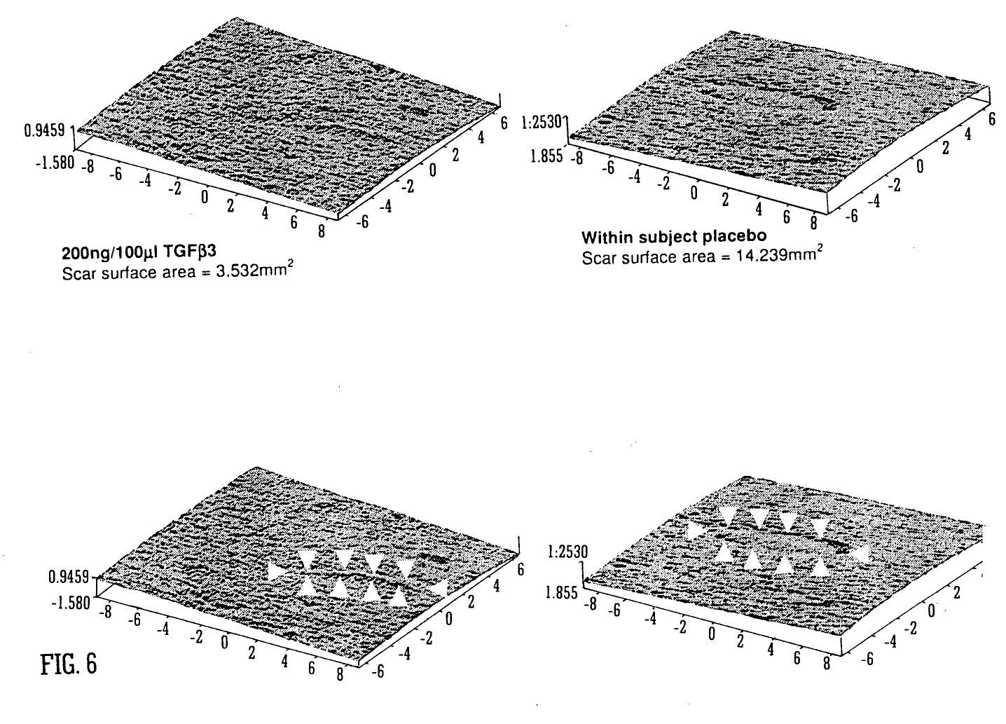

FIG. 6 illustrates 3-dimensional simulations and scar measurements taken from scars formed on healing of wounds treated with either TGF-β3 controls or placebo.

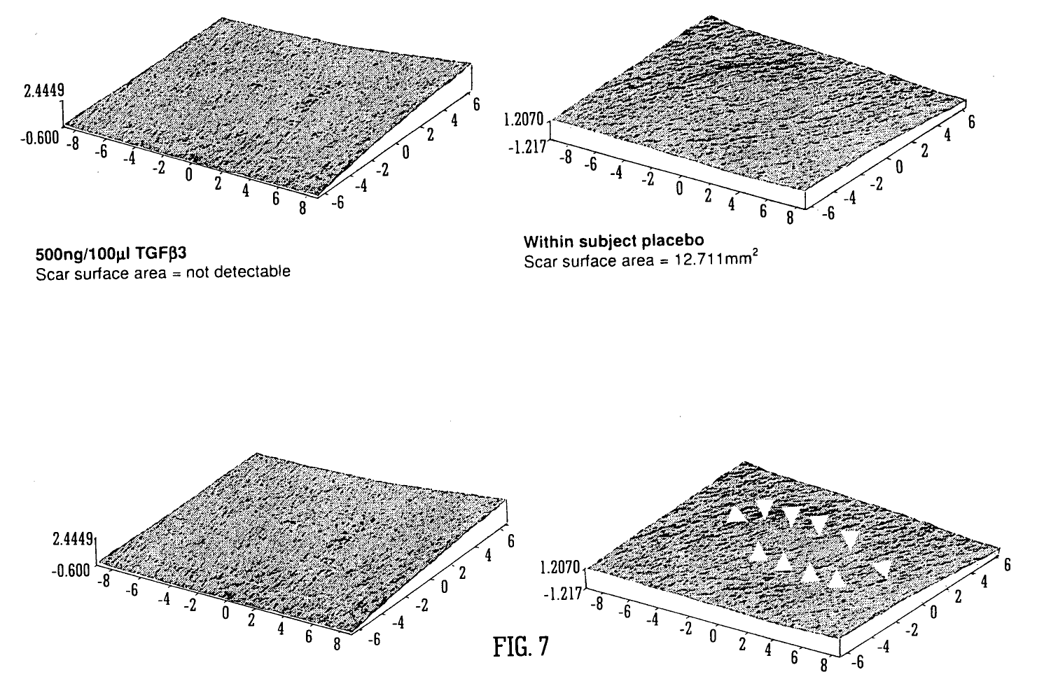

FIG. 7 illustrates 3-dimensional simulations and scar measurements taken from scars formed on healing of wounds treated with either TGF-β3 or with placebo.

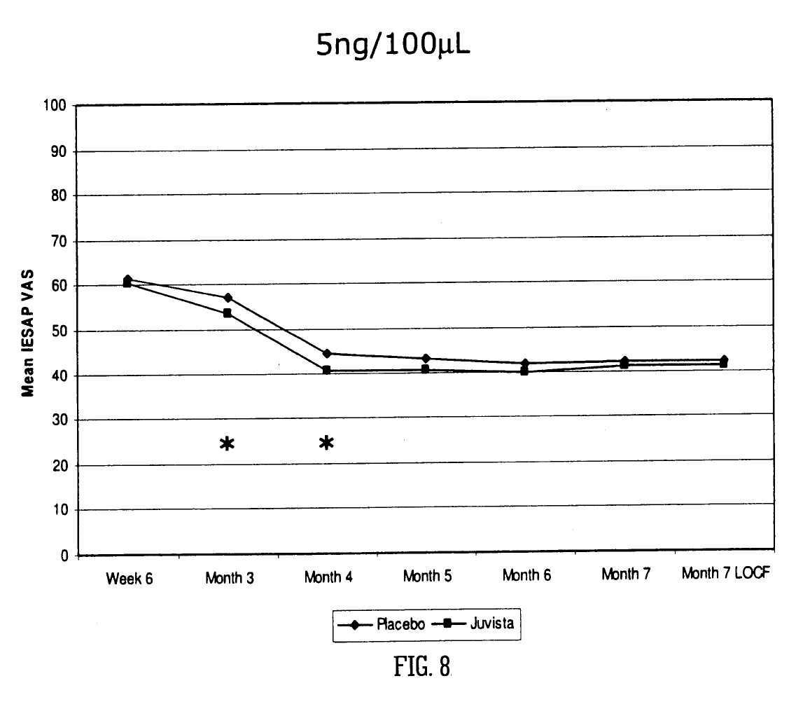

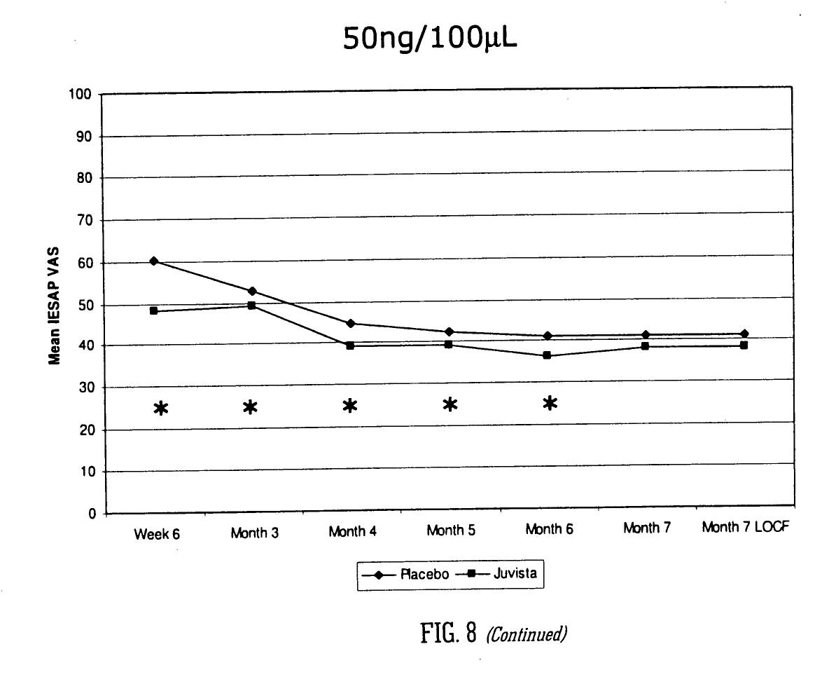

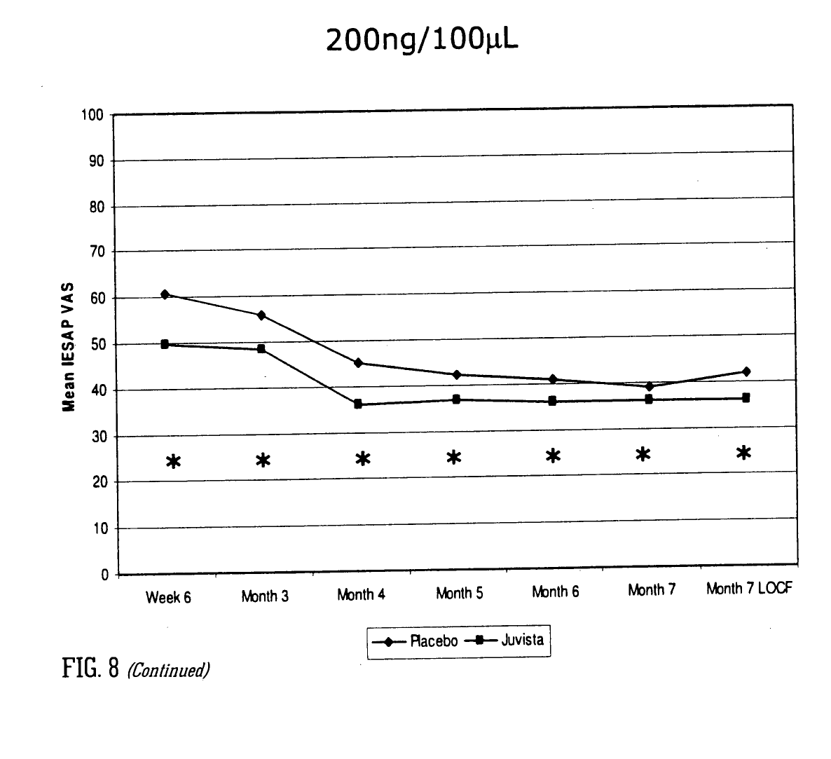

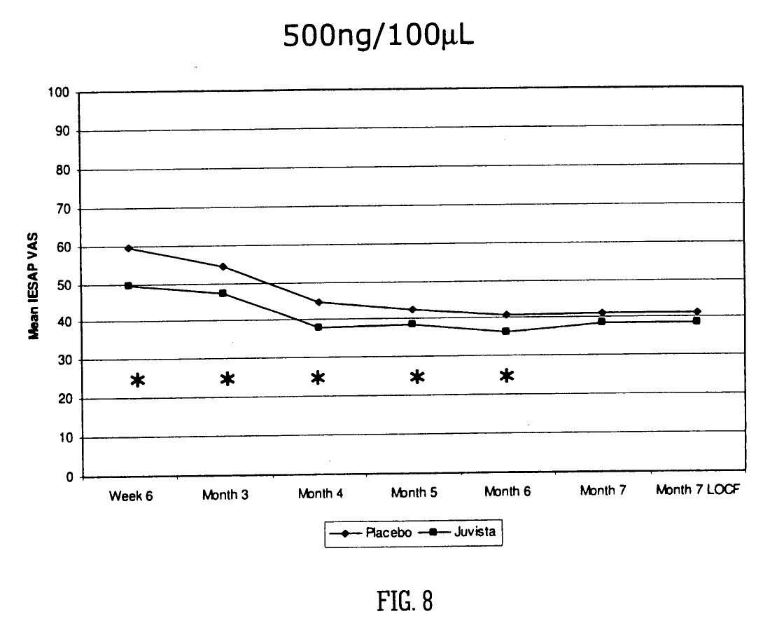

FIG. 8 compares the magnitude of inhibition of scarring achieved over time in control treated scars formed on healing of wounds treated with one of four experimental regimes using TGF-β3 (administered in an amount of 5 ng, 50 ng, 200 ng or 500 ng per centimetre in each of two incidences of treatment separated by approximately one hour).

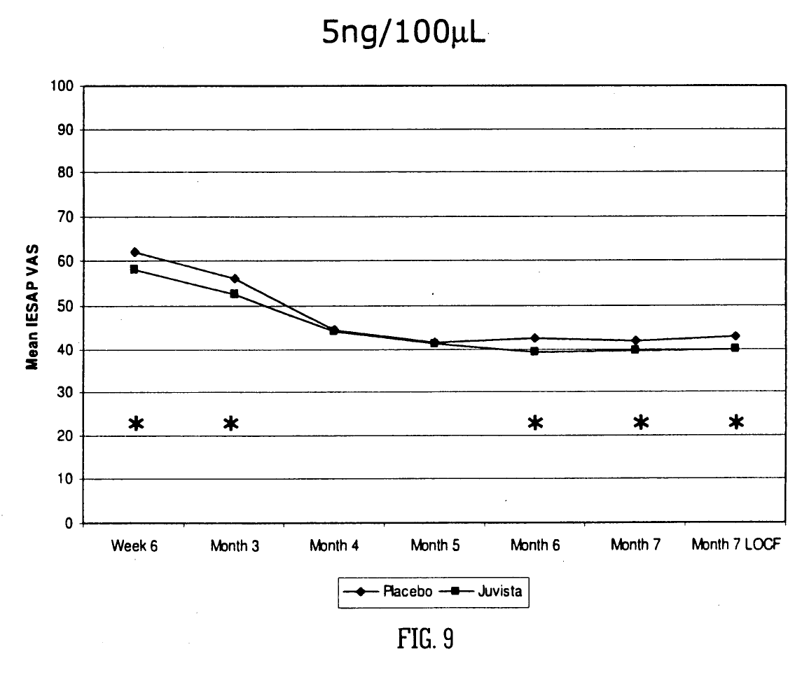

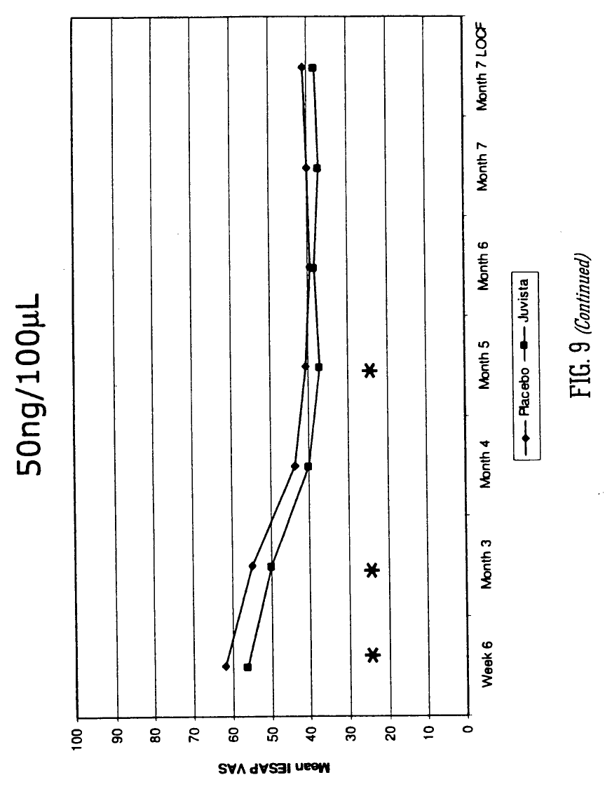

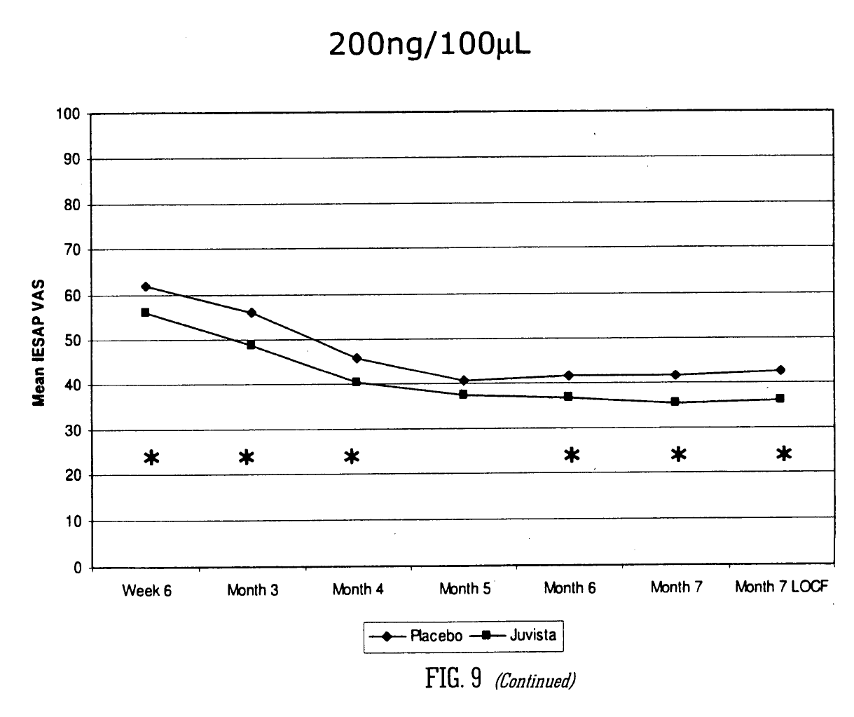

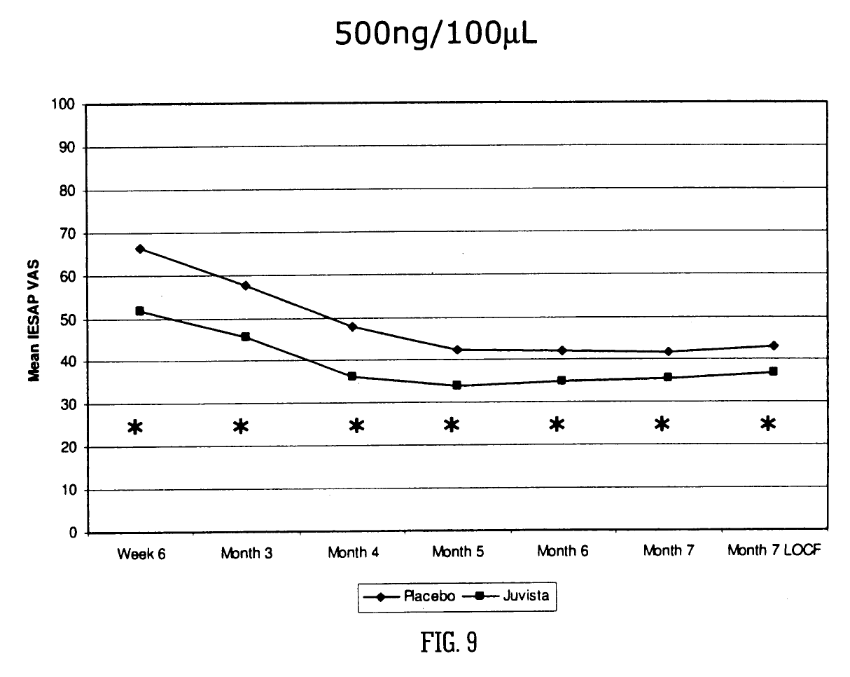

FIG. 9 compares the magnitude of inhibition of scarring achieved over time in control treated scars formed on healing of wounds treated with one of four experimental regimes using TGF-β3 (administered in an amount of 5 ng, 50 ng, 200 ng or 500 ng per centimetre in each of two incidences of treatment separated by approximately 24 hours).

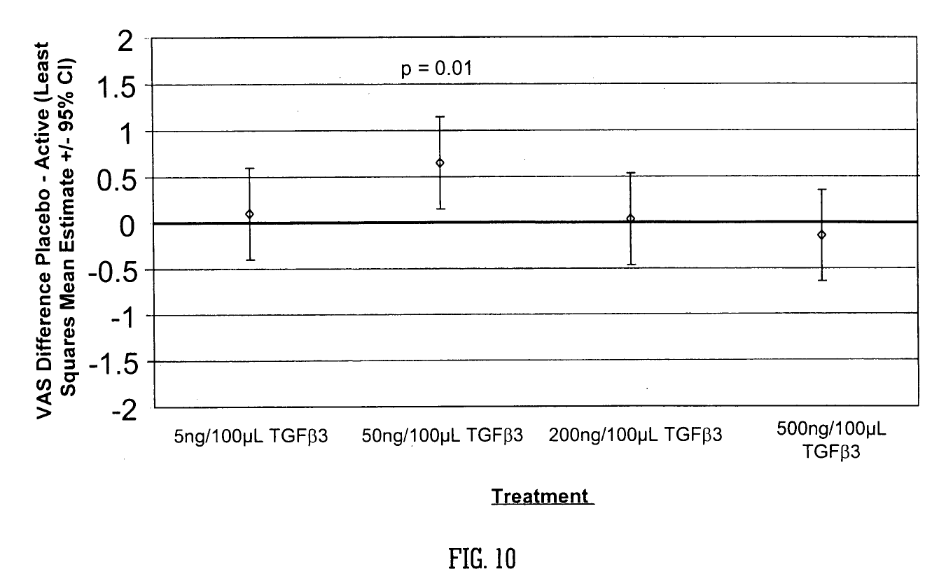

FIG. 10 illustrates a “bell-shaped” dose response curve in a rat model of scar formation in response to different doses of TGF-β3. TGF-β3 was provided to wounds via two injections of TGF-β3 separated by approximately 24 hours. The amount of TGF-β3 provided in each injection was the same in each incidence of treatment.

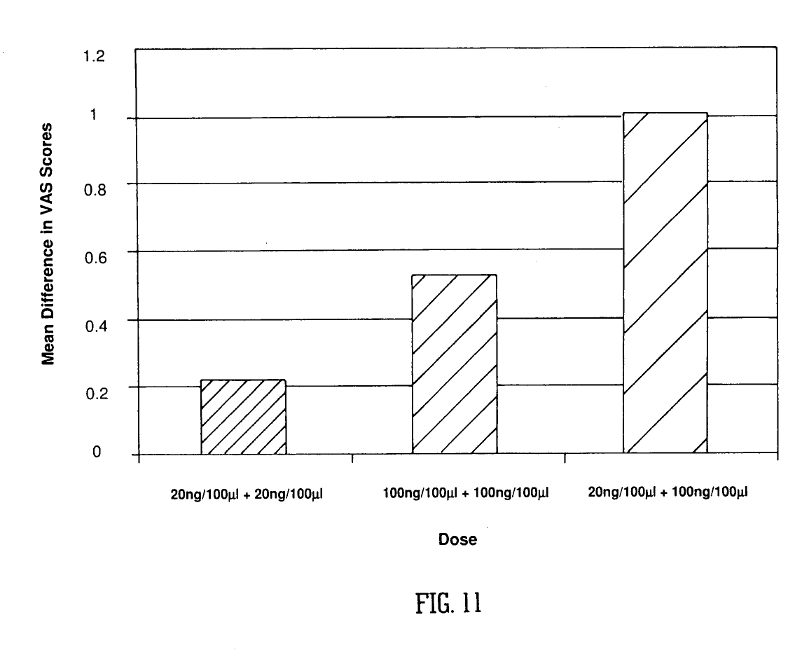

FIG. 11 compares the magnitude of inhibition of scarring achieved on healing of control treated wounds (each subject to two incidences of treatment, in which the amount of TGF-β3 administered remains constant between incidences of treatment) and on healing of wounds treated in accordance with the present invention.

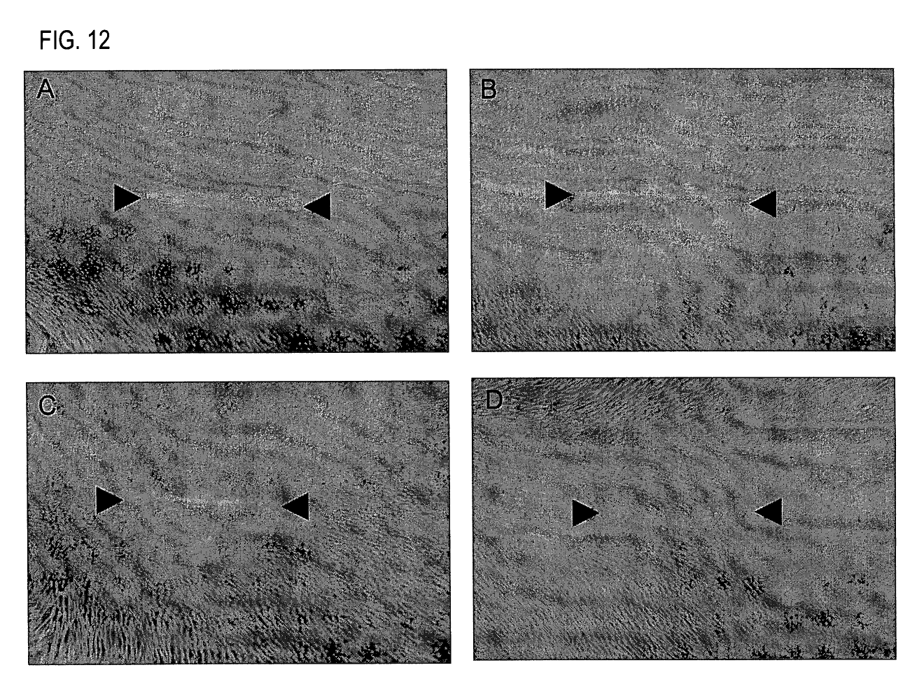

FIG. 12 shows representative images of scars produced on healing of placebo treated wounds (provided with diluent control in two incidences of treatment), control treated wounds (each subject to two incidences of treatment, in which the amount of TGF-β3 administered remains constant between incidences of treatment) and scars produced on healing of wounds treated in accordance with the present invention.

FIG. 13 shows photographs illustrating preferred routes of administration that may be used to provide TGF-β3 to a body site at which it is wished to inhibit scarring in accordance with the present invention. Panel A shows administration of a single injection of a composition comprising TGF-β3 at a site to be wounded. This injection has raised a bleb that covers the site where the wound will be formed (between the two inner dots) and covers an area that extends beyond the intended wound site (the area bounded by the outer dots). Panel B shows the administration of a composition comprising TGF-β3 along a future wound margin. The solid line illustrates the site where a wound is to be formed, and sites at which TGF-β3 may be administered are shown by the dots that surround the future wound. Panels C and D illustrate administration of compositions comprising TGF-β3 to the margins of existing wounds (which have been closed with sutures).

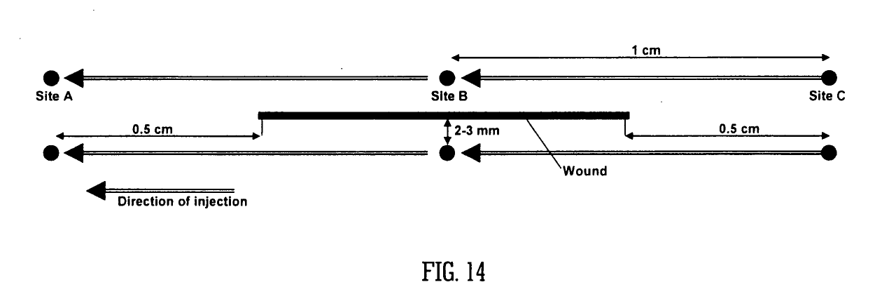

FIG. 14 illustrates a preferred method by which intradermal injections may be used for the administration of TGF-β3 in accordance with the present invention. A hypodermic needle through which TGF-β3 is to be administered is inserted intradermally at site B and advanced to site A (separated from site B by a distance of 1 cm). 100 μl of the composition is then administered evenly between sites A and B as the needle is withdrawn. The needle is then inserted intradermally at site C, advanced in the direction of site B, and the dosing process repeated. When administration to one margin of the wound has been completed, administration may then be repeated on the other margin.

### EXPERIMENTAL RESULTS

FIG. 1

FIG. 1 illustrates data from a clinical trial conducted by the inventors to generate a dose response curve indicative of the anti-scarring effect achieved using various different doses of TGF-β3 administered in a single incidence of treatment. Either TGF-β3 or placebo were administered as a single intradermal injection to a 1 centimetre experimental wound. The figure displays the treatment effect with TGFβ3 as least square means and 95% confidence intervals from an analysis of variance (ANOVA) with site as a factor. To test the treatment effect, ToScar of the TGFβ3 scar was subtracted from the anatomically matched Placebo ToScar on the other arm on each subject. ToScar was calculated as the sum of VAS scores (mm) from week 6 and months 3, 4, 5, 6 and 7. The scars were scored by an independent lay panel at 6 time points after dosing (week 6, months 3, 4, 5, 6 and 7) using a 100 mm VAS line.

FIG. 1 illustrates that scarring is effectively inhibited by a single application of 50 ng, 200 ng or 500 ng/100 μl TGFβ3 per cm of wound margin. The level of improvement displays a typical bell-shaped dose-response curve with maximum improvement (average>50 mm scar improvement in TGFβ3 treated wounds) observed at the 200 ng/100 μl dose, with a reduction in drug efficacy towards the top of the dose range i.e. 500 ng/100 μl per cm of wound margin

FIG. 2

FIG. 2 illustrates data from a clinical trial conducted by the inventors. In this study TGFβ3 and Placebo were each administered in two separate incidences of treatment (by means of two intradermal injections). However, unlike the methods of the present invention, the first incidence of treatment took place immediately prior to wounding but the second incidence of treatment occurred immediately after wound closure, i.e., both doses being administered within approximately 1 hour of one another (the first ten to thirty minutes prior to wounding, and the second ten to thirty minutes post-wounding), and the amount of TGF-β3 provided was the same in each incidence of treatment. The figure displays the treatment effect with TGFβ3 as least square means and 95% confidence intervals from an analysis of variance (ANOVA) with site as a factor. To test the treatment effect, ToScar of the TGFβ3 scar was subtracted from the anatomically matched Placebo ToScar on the other arm on each subject. ToScar was calculated as the sum of VAS scores (mm) from week 6 and months 3, 4, 5, 6 and 7. The scars were scored by an independent lay panel at 6 time points after dosing (week 6, months 3-7) using a 100 mm VAS line.

FIG. 2 illustrates that scarring is effectively inhibited by two applications of 5 ng, 50 ng, 200 ng and 500 ng/100 μl TGFβ3 per cm of wound margin, prior to and immediately after wound closure (i.e. both doses within approximately 1 hour). The level of improvement displays a typical bell-shaped dose-response curve with maximum improvement (average>40 mm scar improvement in TGFβ3 treated wounds) observed at the 200 ng/100 μl dose, with a reduction in drug efficacy towards the top of the dose range i.e. 500 ng/100 μl per cm of wound margin. The degree of improvement and dose-response curve with TGFβ3 treatment given twice (within approximately 1 hour) is comparable to that for TGFβ3 given once (see FIG. 1), though over all the degree to which scarring is inhibited is slightly less than for the single administration regime. This illustrates that repeated administration of TGF-β3 (other than in the methods described in the present invention) does not necessarily lead to a greater inhibition of scarring, and if anything may somewhat diminish the anti-scarring efficacy of this compound.

FIG. 3

FIG. 3 shows comparative data generated by the inventors in a human study. In this study control treatments using TGFβ3 and Placebo were administered in two incidences of treatment (each by intradermal injection), the first prior to wounding and the second approximately 24 hours after wounding. The figure displays the treatment effect with TGFβ3 as least square means and 95% confidence intervals from an analysis of variance (ANOVA) with site as a factor. To test the effect of control treatment with TGF-β3, ToScar of the TGFβ3 control scar was subtracted from the anatomically matched Placebo ToScar on the other arm on each subject. ToScar was calculated as the sum of VAS scores (mm) from week 6 and months 3, 4, 5, 6 and 7. The scars were scored by an independent lay panel at 6 time points after dosing (week 6, months 3, 4, 5, 6 and 7) using a 100 mm VAS line.

FIG. 3 illustrates that scarring is effectively inhibited by two applications of 5 ng, 50 ng, 200 ng and 500 ng/100 μl TGFβ3 per cm of wound margin, prior to and at approximately 24 hours post-wounding. Of these experimental methods of treatment, the method in which 500 ng TGF-β3 is administered in two incidences of treatment separated by 24 hours is notably more effective than the others.

FIG. 4

FIG. 4 shows representative macroscopic images from three subjects illustrating the different extents to which scarring may be inhibited using different TGFβ3 control treatment regimes. The macroscopic images are from within subject scars produced on healing of placebo treated and TGFβ3 control treated wounds (dosed twice with 50 ng, 200 ng or 500 ng/100 μl TGFβ3 per cm of wound margin in two incidences of treatment approximately 24 hours apart) in a clinical trial conducted by the inventors. The same amount of TGF-β3 was administered in each incidence of treatment, and the amounts used are shown in the captions (50 ng/100 μl TGFβ3 per cm of wound margin shown top left, with placebo from the same subject top right; 200 ng/100 μl TGFβ3 per cm of wound margin shown middle left, with placebo from the same subject middle right; and 500 ng/100 μl TGFβ3 per cm of wound margin shown bottom left, with placebo from the same subject bottom right).

The wound receiving control TGF-β3 treatment with the highest dose (bottom left) can be seen to benefit from the greatest inhibition of scarring achieved.

FIG. 5

FIG. 5 shows 3-dimensional simulations and scar measurements obtained from profilometry analysis of silicone moulds from scars produced on healing of placebo treated and TGFβ3 control treated wounds (dosed twice with 100 μl of 50 ng/100 μl TGFβ3 or 100 μl placebo per cm of wound margin approximately 24 hours apart) in a clinical trial conducted by the inventors. Note that this is not a method of treatment in accordance with the invention, but (along with FIG. 6) serves to provide comparative data illustrating the surprising effectiveness of methods of treatment in accordance with the invention.

The top panel shows the original 3-dimensional simulations and for clarity the bottom panel illustrates the boundaries of the scars demarcated by white arrowheads, with the remaining area of the image being normal skin surrounding the scar. A range of quantitative parameters for each scar were analysed by profilometry and demonstrated a 30.21% reduction in scar surface area with TGFβ3 treatment compared to placebo (TGFβ3 treated wound scar surface area=12.823 mm²; placebo treated wound scar surface area=18.375 mm²).

FIG. 6

FIG. 6 shows 3-dimensional simulations and scar measurements obtained from profilometry analysis of silicone moulds from scars produced on healing of placebo treated and TGFβ3 control treated wounds (dosed twice with 100 μl of 200 ng/100 μl TGFβ3 or 100 μl placebo per cm of wound margin approximately 24 hours apart) in a clinical trial conducted by the inventors. As with the results shown in FIG. 6, this does not constitute a method of treatment in accordance with the invention, but instead serves to provide comparative data illustrating the surprising effectiveness of methods of treatment in accordance with the invention.

The top panel shows the original 3-dimensional simulations and for clarity the bottom panel illustrates the boundaries of the scars demarcated by white arrowheads, with the remaining area of the image being normal skin surrounding the scar. A range of quantitative parameters for each scar were analysed by profilometry and demonstrated a 75.19% reduction in scar surface area with TGFβ3 treatment compared to placebo (TGFβ3 treated wound scar surface area=3.532 mm²; placebo treated wound scar surface area=14.239 mm²). Profilometry analysis also demonstrated a reduction in scar raised volume with TGFβ3 treatment of 73.33% compared to placebo treatment (TGFβ3 treated wound scar raised volume=0.0008 mm³; placebo treated wound scar raised volume=0.003 mm³).

FIG. 7

FIG. 7 shows 3-dimensional simulations and scar measurements obtained from profilometry analysis of silicone moulds from scars produced on healing of placebo treated and TGFβ3 control treated wounds (dosed twice with 100 μl of 500 ng/100 μl TGFβ3 or 100 μl placebo per cm of wound margin in two incidences of treatment providing equal amounts of TGF-β3 approximately 24 hours apart from one another).

The top panel shows the original 3-dimensional simulations and for clarity the bottom panel illustrates the boundaries of the scars demarcated by white arrowheads, with the remaining area of the image being normal skin surrounding the scar. Maximal inhibition of scarring achieved in this study is observed in response to treatment with two relatively high doses of TGF-β3. While this approach may be effective to inhibit scarring the cost associated with such treatment regimes will be higher than for methods of treatment in accordance with the present invention (where effective inhibition of scarring may be achieved while using a smaller overall quantity of TGF-β3).

FIG. 8

FIG. 8 illustrates data from a clinical trial conducted by the inventors in which either TGF-β3 or placebo were administered in two incidents of treatment (each comprising administration of the test substance by intradermal injection), the first incidence occurring prior to wounding and the second immediately after wound closure, i.e., both doses of TGF-β3 being the same as one another, and administered within approximately 1 hour (10-30 mins prior to wounding and 10-30 mins post wounding). It will be recognised that the experimental methods of treatment, the results of which are shown in FIG. 8, do not represent methods of treatment in accordance with the present invention, but are instead alternative (therapeutically effective) methods of treatment that illustrate the surprising efficacy of the methods of the invention.

FIG. 8 displays the treatment effect with TGF-β3 (here labelled “Juvista”) and placebo as mean visual analogue scale (VAS) scores (mm). The scars were scored by an independent lay panel at 6 time points after dosing (week 6 and months 3-7) using a 100 mm VAS line.

FIG. 8 illustrates that scarring is inhibited by two applications of 100 μl of 5 ng, 50 ng, 200 ng and 500 ng/100 μl TGF-β3 per cm of wound margin administered prior to and immediately after wound closure (i.e. both doses within approximately 1 hour). The level of improvement is dose responsive and typically is first evident at early time points (week 6 onwards) and is maintained throughout the assessment period (i.e., up to 7 months in this study).

* indicates significant difference (p<0.05) between scarring resulting from healing of wounds provided with the TGF-β3 control treatment and those provided with placebo treatment

FIG. 9

FIG. 9 illustrates data from a further clinical trial conducted by the inventors comparing therapeutically effective anti-scarring treatments using TGF-β3.

TGFβ3 and Placebo were administered by means of intradermal injection in two incidences of treatment, the first prior to wounding and the second approximately 24 hours later. The amount of TGF-β3 provided did not alter between incidences of treatment, and hence this study does not constitute treatment in accordance with the present invention. The figure displays the treatment effect with TGFβ3 (once more labelled “Juvista”) and placebo as mean visual analogue scale (VAS) scores (mm). The scars were scored by an independent lay panel at 6 time points after dosing (week 6, months 3-7) using a 100 mm VAS line.

FIG. 9 illustrates that scarring is inhibited by two applications of 100 μl of 5 ng, 50 ng, 200 ng or 500 ng/100 μl TGFβ3 per cm of wound margin administered prior to wounding and at approximately 24 hour post-wounding. The level of improvement is dose responsive and typically is first evident at early time points (week 6 onwards) and is maintained throughout the assessment period (i.e., up to 7 months in this study). Surprisingly the magnitude of effect is much larger than expected from previous data. It can be seen that the method of the invention (in which 500 ng of TGF-β3 is provided per centimetre of the body site treated in each incidence of treatment) is surprisingly more effective than the other methods of treatment (which are themselves still therapeutically effective).

* indicates significant difference (p<0.05) between scarring resulting from healing of wounds provided with the TGFβ3 control treatment and those provided with Placebo treatment

FIG. 10

FIG. 10 illustrates that the TGF-β3 “bell-shaped” dose response curve observed in human subjects is also found in experimental animals. Here, TGF-β3 was provided to experimental rat wounds, in two incidences of treatment separated by 24 hours (the first incidence of treatment occurring at, or around, the time of wounding). The amount of TGF-β3 administered per centimetre of wound in each incidence of treatment is shown on the X-axis (5 ng/cm, 50 ng/cm, 200 ng/cm or 500 ng/cm).

As can be seen, repeated treatment with low doses of TGF-β3 or with high doses of TGF-β3 brought about little inhibition of scarring.

FIG. 11

A rat experimental model of wound healing and scarring was used to illustrate the inhibition of scarring that may be achieved using the medicaments and methods of the present invention, as compared to untreated controls, or control treatments with TGF-β3 in which the amount of TGF-β3 administered does not increase between first and second incidences of treatment.

FIG. 11 is a graph comparing the mean differences between macroscopic VAS scores of scars formed on healing of 1 cm incisional rat wounds treated with a diluent control (“placebo treated wounds”), and scars formed on healing of wounds provided with one of the following regimes:
i) TGF-β3 control treatment using 20 ng TGF-β3 per centimetre;
ii) TGF-β3 control treatment using 100 ng TGF-β3 per centimetre; or
iii) TGFβ3 treatment in accordance with the present invention.

In each case the wounds were subject to two incidences of treatment, the first prior to wounding and the second approximately 24 hours later.

Placebo treated control wounds were provided with two incidences of treatment, each of which consisted of administration of a diluent. These placebo treated wounds provide a baseline value for scarring, with reference to which scar inhibition produced by TGF-β3 treatments may be determined. “Control treated wounds” were provided with two incidences of treatment, each comprising injections of TGF-β3 at either 20 ng/100 μl or 100 ng/100 μl (the same concentration of TGF-β3 being injected in each incidence of treatment). The “treated wounds” were provided with an escalating dose regimen in accordance with the present invention, in which the first incidence of treatment comprised an injection of 20 ng/100 μl TGFβ3, while the second incidence of treatment comprised an injection of 100 ng/100 μl TGFβ3.

Each animal received two wounds, and these were arranged so that the wounds of each animal included placebo treated wounds, as well as either treated wounds (examples treated with TGFβ3 in accordance with the invention), or control treated wounds (receiving control treatment with TGF-β3 at the same dose in each incidence of treatment). This permits comparison between scars formed on healing of placebo treated wounds and treated or control treated wounds within the same subject. This study design allows intra-subject variability to be reduced when assessing the anti-scarring effect of TGFβ3 treatment (either control treatment or treatment in accordance with the invention).

Scars were assessed, and VAS scores produced, 70 days after wounding.

In keeping with the results reported in FIG. 10 above, control treated wounds (dosed twice with either 20 ng/100 μl or 100 ng/100 μl TGFβ3) displayed a reduction in scarring as compared to control untreated wounds receiving placebo. This is not surprising, since the amounts of TGF-β3 are in the region shown to be most effective in the “bell-shaped” distribution in this model. However, it is a surprising finding that wounds dosed in accordance with the methods of the invention (in which a larger amount of TGFβ3 is provided in the second incidence of treatment than the therapeutically effective amount administered in the first incidence of treatment) displayed a much larger magnitude of effect in terms of the inhibition of scarring achieved on healing of the wound. The anti-scarring effect of dosing with 20 ng/100 μl TGFβ3 followed by 100 ng/100 μl TGFβ3 is a much larger synergistic effect than that which would be expected by an additive anti-scarring effect achieved in line with the results of either 20 ng/100 μl or 100 ng/100 μl TGFβ3 dosed twice.

The results illustrate that the inhibition of scarring observed on healing of wounds treated with the methods of the invention is much greater than that observed on healing of wounds treated using alternative treatment regimens involving the administration of TGF-β3 in two incidences of treatment providing equal doses of TGF-β3.

FIG. 12

FIG. 12 shows representative images of the macroscopic appearance of scars produced by the studies described in connection with FIG. 11 above. These images of the scars were collected 70 days post wounding, and the arrow heads shown mark the ends of the scars.

The scars shown are those formed on healing of 1 cm incisional rat wounds provided with two incidents of treatment, 24 hours apart, with either placebo (to provide placebo treated control wounds) or TGF-β3 (to produce either treated wounds, receiving an escalating dosage regime in accordance with the present invention, or control treated wounds).

Representative images of scars produced on the healing of control placebo treated wounds are shown in Panel A. Panel B illustrates scars produced on healing of TGFβ3 control treated wounds provided with two incidents of treatment, each comprising injection of 20 ng/100 μl TGFβ3. Panel C illustrates scars produced on healing of TGFβ3 control treated wounds provided with two incidents of treatment, each comprising injection of 100 ng/100 μl TGFβ3. The scars shown in Panel D were produced on healing of wounds treated in accordance with the present invention. In a first incidence of treatment they were injected with 20 ng/100 μl TGFβ3, and in a second incidence of treatment were injected with 100 ng/100 μl TGFβ3.

The images illustrate that scars resulting from wounds treated with TGFβ3 are reduced in comparison to placebo treated wounds, in that they exhibit reduced width, are less white (a reduction in hypopigmentation) and blend better with the surrounding skin. The fact that the control TGF-β3 treated wounds exhibit a reduction in scarring is consistent with the effects observed in the generation of the dose response curve shown above. As reported in connection with FIG. 11, the wounds treated with an escalating dose regimen of 20 ng/100 μl TGFβ3 prior to wounding followed by an injection of 100 ng/100 μl TGFβ3 approximately 24 hours later, display the greatest inhibition in scarring, with resultant scars which more closely approximate the surrounding unwounded skin than do scars produced on the healing of wounds treated with other treatment regimens.

Sequence Information

TGF-β3 (Sequence ID No. 1)
ALDTNYCFRNLEENCCVRPLYIDFRQDLGWKWVHEPKGYYANFCSGPCP
YLRSADTTHSTVLGLYNTLNPEASASPCCVPQDLEPLTILYYVGRTPKV
EQLSNMVVKSCKCS
Sequence ID No. 2-DNA encoding wild-
type human TGF-β3
GCT TTG GAC ACC AAT TAC TGC TTC CGC AAC TTG GAG
GAG AAC TGC TGT GTG CGC CCC CTC TAC ATT GAC TTC
CGA CAG GAT CTG GGC TGG AAG TGG GTC CAT GAA CCT
AAG GGC TAC TAT GCC AAC TTC TGC TCA GGC CCT TGC
CCA TAC CTC CGC AGT GCA GAC ACA ACC CAC AGC ACG
GTG CTG GGA CTG TAC AAC ACT CTG AAC CCT GAA GCA
TCT GCC TCG CCT TGC TGC GTG CCC CAG GAC CTG GAG
CCC CTG ACC ATC CTG TAC TAT GTT GGG AGG ACC CCC
AAA GTG GAG CAG CTC TCC AAC ATG GTG GTG AAG TCT
TGT AAA TGT AGC

1. A method of inhibiting scarring formed on healing of a wound, the method comprising treating a body site in which scarring is to be inhibited:
in a first incidence of treatment providing to each centimetre of wound margin, or each centimetre of a site at which a wound is to be formed a first therapeutically effective amount of TGF-β3; and
in a second incidence of treatment, occurring after a wound is formed and between 8 and 48 hours after the first incidence of treatment, providing to said wound a therapeutically effective amount of TGF-β3 that is larger than the therapeutically effective amount of TGF-β3 provided in the first incidence of treatment.

2. The method according to claim 1, wherein the TGF-β3 is provided by means of a local injection.

3. The method according to claim 2, wherein the first incidence of treatment is provided at a site where a wound is to be formed and the local injection is to be administered substantially along the midline of the wound to be formed.

4. The method according to claim 2, wherein the first incident of treatment is provided to a site at which a wound is to be formed and wherein a local injection is administered on each of the margins of the wound to be formed.

5. The method according to claim 2, wherein the first and or second incidence of treatment is provided to a wound margin and the local injection is administered at a location within half a centimetre of the wound margin

6. The method according to claim 1, wherein the first and/or second incidence of treatment comprises providing the TGF-β3 to a region extending at least half a centimetre beyond each end of the wound.

7. A method of inhibiting scarring formed on healing of a wound, the method comprising treating a body site in which scarring is to be inhibited:
in a first incidence of treatment providing to each centimetre of a site where a wound is to be formed a first therapeutically effective amount of TGF-β3; and
in a second incidence of treatment, occurring after a wound is formed and between 8 and 48 hours after the first incidence of treatment, providing to said wound a therapeutically effective amount of TGF-β3 that is larger than the therapeutically effective amount of TGF-β3 provided in the first incidence of treatment.

8. A method of inhibiting scarring formed on healing of a wound, the method comprising treating a body site in which scarring is to be inhibited:
in a first incidence of treatment providing to each centimetre of wound margin, or each centimetre of future wound margin, a first therapeutically effective amount of TGF-β3; and
in a second incidence of treatment, occurring after a wound is formed and between 8 and 48 hours after the first incidence of treatment, providing to said wound a therapeutically effective amount of TGF-β3 that is larger than the therapeutically effective amount of TGF-β3 provided in the first incidence of treatment.

9. A method according to claim 1, further comprising a third or further incidence of treatment.

10. A method according to claim 9, wherein the amount of TGF-β3 provided in the third or further incidence of treatment is substantially the same as the amount provided in the second incidence of treatment.

11. A method according to claim 9, wherein the therapeutically effective amount of TGF-β3 provided in the third or further incidence of treatment, is larger than the amount of TGF-β3 provided in the preceding incident of treatment.

12. A method according to claim 11, wherein the amount of TGF-β3 provided per centimetre of wounding in the second, or further, incidence of treatment is at least 100 ng larger than the amount provided in the preceding incident of treatment.

13. A method according to claim 12, wherein the amount of TGF-β3 provided per centimetre of wounding in the second, or further, incidence of treatment is at least 500 ng larger than the amount provided in the preceding incident of treatment.

14. A method according to any preceding claim 1, wherein the amount of TGF-β3 provided per centimetre of wound margin, or potential wound margin, in the first incidence of treatment is approximately 100 ng.

15. A method according to claim 1, wherein the amount of TGF-β3 provided per centimetre of wound margin, or potential wound margin, in the first incidence of treatment is approximately 200 ng.

16. A method according to claim 1, wherein the incidences of treatment are separated by approximately 24 hours.

17. A method according to claim 1, wherein the wound is a skin wound.

18. The method according to claim 1, where the wound is a wound of the circulatory system

19. A method according to claim 1, wherein the wound is a result of surgery.

20. A method according to claim 7, wherein the TGF-β3 is provided by local injection administered to the body site.

21. A method according to claim 7, wherein the TGF-β3 is provided in a pharmaceutically acceptable solution, approximately 100 μl of which is administered per centimetre of body site treated.

22. A method according to claim 7, wherein the first incidence of treatment occurs prior to wounding.

23. A method according to claim 22, wherein the first incidence of treatment occurs up to an hour prior to wounding.

24. A method according to claim 7, wherein the first incidence of treatment occurs after wounding.

25. A method according to claim 24, wherein the first incidence of treatment occurs up to two hours after wounding.

26. A method according to claim 7, wherein the first incidence of treatment occurs after wound closure.

27. A method according to claim 26, wherein the first incidence of treatment occurs up to two hours after wound closure.

28. A method of selecting an appropriate treatment regime for inhibiting scarring associated with the healing of a wound, the method comprising:
determining whether an individual in need of such inhibition of scarring will be able to complete a second incidence of treatment occurring between 8 and 48 hours after a first incidence of treatment;
if the individual will be able to complete a second incidence of treatment occurring between 8 and 48 hours after a first incidence of treatment, selecting a treatment regime comprising treating a body site in which scarring is to be inhibited such that:
in a first incidence of treatment providing to each centimetre of wound margin, or each centimetre of a site at which a wound is to be formed a first therapeutically effective amount of TGF-β3; and
in a second incidence of treatment, occurring after a wound is formed and between 8 and 48 hours after the first incidence of treatment, providing to said wound a therapeutically effective amount of TGF-β3 that is larger than the therapeutically effective amount of TGF-β3 provided in the first incidence of treatment; or
if the individual will not be able to complete a second incidence of treatment occurring between 8 and 48 hours after a first incidence of treatment, selecting a treatment regime comprising:
in a single incidence of treatment providing to each centimetre of wound margin, or each centimetre of a site at which a wound is to be formed, in which scarring is to be inhibited an amount of between approximately 150 ng and 349 ng TGF-β3.

29. The use of TGF-β3 as a medicament in treating a wound or site where a wound is to be formed to inhibit scarring, wherein in a first incidence of treatment the medicament is provided such that a first therapeutically effective amount of TGF-β3 is provided to each centimetre of a wound margin or each centimetre of a site at which a wound is to be formed; and wherein in a subsequent incidence of treatment the medicament is provided such that a larger therapeutically effective amount of TGF-β3 is provided to each centimetre of a wound margin between 8 hours and 48 hours after the previous incidence of treatment.

30. TGF-β3 used according to claim 29, wherein the medicament is an injectable medicament.

31. TGF-β3 used according to claim 30, wherein the medicament is for intradermal injection.

32. TGF-β3 used according to claim 29, wherein the medicament is formulated such that the requisite amount of TGF-β3 is provided in a 100 μl volume of the medicament.

33. TGF-β3 for use as a medicament in treating a wound or site where a wound is to be formed to inhibit scarring, wherein in a first incidence of treatment the medicament is provided such that a first therapeutically effective amount of TGF-β3 is provided to each centimetre of a wound margin or each centimetre of a site at which a wound is to be formed; and wherein in a subsequent incidence of treatment the medicament is provided such that a larger therapeutically effective amount of TGF-β3 is provided to each centimetre of a wound margin between 8 hours and 48 hours after the previous incidence of treatment.

34. TGF-β3 for use according to claim 33, wherein the medicament is an injectable medicament.

35. TGF-β3 for use according to claim 34, wherein the medicament is for intradermal injection.

36. TGF-β3 for use according to claim 33, wherein the medicament is formulated such that the requisite amount of TGF-β3 is provided in a 100 μl volume of the medicament.

37. A kit for use in the inhibition of scarring associated with healing of a wound, the kit comprising at least first and second vials comprising TGF-β3 for administration to a wound, or a site where a wound is to be formed, at times between 8 and 48 hours apart from one another.

38. A kit for use in the inhibition of scarring associated with healing of a wound, the kit comprising:
a first amount of a composition containing TGF-β3, this first amount being for administration to a wound, or a site where a wound is to be formed, in a first incidence of treatment;
a second amount of a composition containing TGF-β3, this second amount being for administration to a wound in a second incidence of treatment;
instructions regarding administration of the first and second amounts of the composition at times between 8 and 48 hours apart from one another, and in a manner such that a larger therapeutically effective dose of TGF-β3 is administered to the wound in the second incidence of treatment than was administered in the first incidence of treatment.

39. A kit according to claim 38, wherein the first and second amounts of a composition respectively comprise different first and second compositions, wherein the second composition contains TGF-β3 at a greater concentration than does the first composition

40. A kit according to claim 38, wherein the first and second compositions contain TGF-β3 at substantially equal concentrations, and the instructions indicate that the volume of the second composition administered in the second incidence of treatment should be larger than the volume of the first composition administered in the first incidence of treatment.

41. A method according to claim 7, further comprising a third or further incidence of treatment.

42. A method according to claim 41, wherein the amount of TGF-β3 provided in the third or further incidence of treatment is substantially the same as the amount provided in the second incidence of treatment.

43. A method according to claim 41, wherein the therapeutically effective amount of TGF-β3 provided in the third or further incidence of treatment, is larger than the amount of TGF-β3 provided in the preceding incident of treatment.

44. A method according to claim 43, wherein the amount of TGF-β3 provided per centimetre of wounding in the second, or further, incidence of treatment is at least 100 ng larger than the amount provided in the preceding incident of treatment.

45. A method according to claim 44, wherein the amount of TGF-β3 provided per centimetre of wounding in the second, or further, incidence of treatment is at least 500 ng larger than the amount provided in the preceding incident of treatment.

46. A method according to claim 7, wherein the amount of TGF-β3 provided per centimetre of wound margin, or potential wound margin, in the first incidence of treatment is approximately 100 ng.

47. A method according to claim 7, wherein the amount of TGF-β3 provided per centimetre of wound margin, or potential wound margin, in the first incidence of treatment is approximately 200 ng.

48. A method according to claim 7, wherein the incidences of treatment are separated by approximately 24 hours.

49. A method according to claim 7, wherein the wound is a skin wound.

50. The method according to claim 7, where the wound is a wound of the circulatory system

51. A method according to claim 7, wherein the wound is a result of surgery.

52. A method according to claim 8, further comprising a third or further incidence of treatment.

53. A method according to claim 52, wherein the amount of TGF-β3 provided in the third or further incidence of treatment is substantially the same as the amount provided in the second incidence of treatment.

54. A method according to claim 52, wherein the therapeutically effective amount of TGF-β3 provided in the third or further incidence of treatment, is larger than the amount of TGF-β3 provided in the preceding incident of treatment.

55. A method according to claim 54, wherein the amount of TGF-β3 provided per centimetre of wounding in the second, or further, incidence of treatment is at least 100 ng larger than the amount provided in the preceding incident of treatment.

56. A method according to claim 55, wherein the amount of TGF-β3 provided per centimetre of wounding in the second, or further, incidence of treatment is at least 500 ng larger than the amount provided in the preceding incident of treatment.

57. A method according to claim 8, wherein the amount of TGF-β3 provided per centimetre of wound margin, or potential wound margin, in the first incidence of treatment is approximately 100 ng.

58. A method according to claim 8, wherein the amount of TGF-β3 provided per centimetre of wound margin, or potential wound margin, in the first incidence of treatment is approximately 200 ng.

59. A method according to claim 8, wherein the incidences of treatment are separated by approximately 24 hours.

60. A method according to claim 8, wherein the wound is a skin wound.

61. The method according to claim 8, where the wound is a wound of the circulatory system

62. A method according to claim 8, wherein the wound is a result of surgery.

63. A method according to claim 8, wherein the TGF-β3 is provided by local injection administered to the body site.

64. A method according to claim 8, wherein the TGF-β3 is provided in a pharmaceutically acceptable solution, approximately 100 μl of which is administered per centimetre of body site treated.

65. A method according to claim 8, wherein the first incidence of treatment occurs prior to wounding.

66. A method according to claim 65, wherein the first incidence of treatment occurs up to an hour prior to wounding.

67. A method according to claim 8, wherein the first incidence of treatment occurs after wounding.

68. A method according to claim 67, wherein the first incidence of treatment occurs up to two hours after wounding.

69. A method according to claim 8, wherein the first incidence of treatment occurs after wound closure.

70. A method according to claim 69, wherein the first incidence of treatment occurs up to two hours after wound closure.

71. A method according to claim 1, wherein the TGF-β3 is provided in a pharmaceutically acceptable solution, approximately 100 μl of which is administered per centimetre of body site treated.
